(19) United States
(12) Patent Application Publication
(10) Pub. No.: US 2023/0099106 A1
(43) Pub. Date: Mar. 30, 2023

(54) PRODRUGS USEFUL IN ADOPTIVE CELL THERAPY

(71) Applicant: Memorial Sloan Kettering Cancer Center, New York, NY (US)

(72) Inventors: David A. Scheinberg, New York, NY (US); Thomas J. Gardner, New York, NY (US); Derek Tan, New York, NY (US); Jonghan P. Lee, New York, NY (US)

(21) Appl. No.: 17/795,751

(22) PCT Filed: Jan. 28, 2021
(86) PCT No.: PCT/US2021/015424

§ 371 Date: Jul. 27, 2022

Related U.S. Application Data
(60) Provisional application No. 62/967,370, filed on Jan. 29, 2020.

(51) Int. Cl. A61K 31/517; A61K 35/17; A61P 35/00

(52) U.S. Cl. CPC A61K 31/517; A61K 35/17; A61P 35/00

(57) ABSTRACT

Provided herein are prodrug compounds of Formula (I) and compositions thereof useful in methods for adoptive cell therapy.

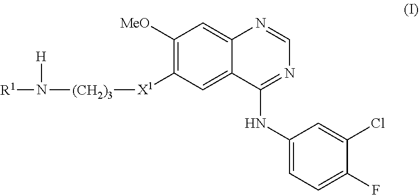

### CROSS-REFERENCE TO RELATED APPLICATIONS

This application claims the benefit of and priority to U.S. Provisional Appl. No. 62/967,370, filed Jan. 29, 2020, incorporated herein by reference in its entirety.

### STATEMENT OF GOVERNMENT SUPPORT

This invention was made with government support under CA055349, CA023766, GM100477 and All 18224 awarded by the National Institutes of Health. The government has certain rights in the invention.

### BACKGROUND

Adoptive and engineered T cell therapies, including chimeric antigen receptor (CAR) T cells, T cell receptor (TCR) engineered T cells, and antigen adopted T cells, have emerged recently as important therapies for cancer. First generation CARs were designed by fusing the scFv to the intracellular signaling domain of the CD3-ζ chain, whereas second generation CARs added CD28-CD80 costimulation in which the CD28 signaling domain was incorporated into the CAR construct (i.e.: “28ζ CAR”) for improved T cell activation and efficacy. However, greater potency and mechanisms to defeat the immunosuppressive tumor microenvironment are still needed for many cancer types. Resistance to the activity of these cells, relapse, and toxicity are still important hurdles to their success. Recent reports of significant toxicities and even deaths after CAR T cell therapy, and TCR T cell therapy, and the continuing incidence of GVHD with its associated short- and long-term morbidity and mortality, call for new methods to potentiate efficacy, and at the same time, to better control the various adoptive cell therapies.

Accordingly, in attending to these the present inventors provided compositions and methods for adoptive cell therapy comprising engineered immune cells that express a prodrug converting enzyme and a receptor that binds to a target antigen, as disclosed in Int'l Appl. No. PCT/US2018/040629 (published as WO2019/006464), Int'l Appl. No. PCT/US2018/040633 (published as WO2018/040633), Int'l Appl. No. PCT/US2018/040640 (published as WO2019/006468) and Int'l Appl. No. PCT/US2018/040639 (published as WO 2019/006467).

### SUMMARY

The present technology provides prodrug compounds useful with and/or as part of compositions and in methods for adoptive cell therapy as disclosed in Int'l Appl. No. PCT/US2018/040629 (published as WO2019/006464), Int'l Appl. No. PCT/US2018/040633 (published as WO2018/040633), Int'l Appl. No. PCT/US2018/040640 (published as WO2019/006468) and Int'l Appl. No. PCT/US2018/040639 (published as WO 2019/006467).

Thus, in an aspect, a prodrug compound of Formula I is provided

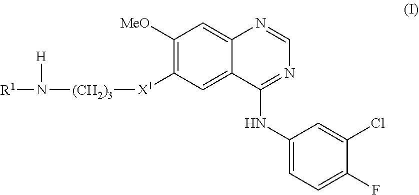

or a pharmaceutically acceptable salt, and/or solvate thereof, where X¹ is O or NH, R¹ is

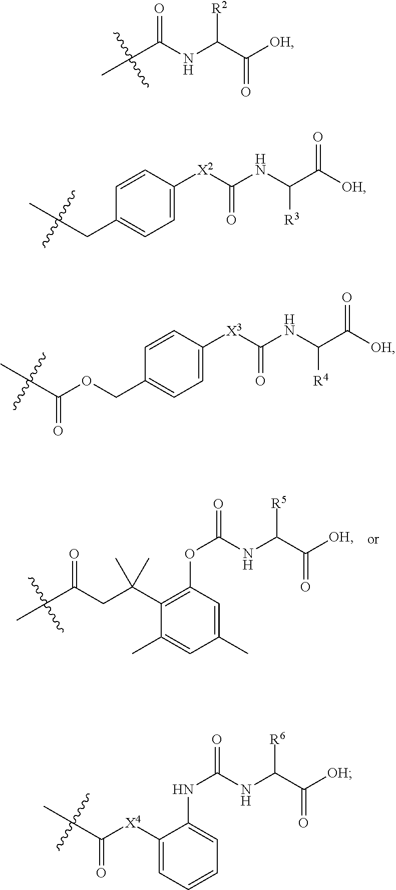

X² and X³ are each independently O or NH; X⁴ is O, NH, CH₂, or C(Me)₂; and R², R³, R⁴, R⁵, and R⁶ are each independently H, —(CH₂)₃—NH(NH)—NH₂, —(CH₂)₃—NH₂, —(CH₂)₄—NH₂, —CH₂—C(O)OH, —(CH₂)₂—C(O)OH,

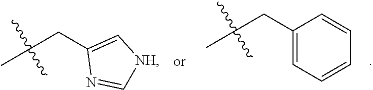 [SMILES: *Cc1c[nH]cn1 ; *Cc1ccccc1]

In any embodiment herein, it may be R¹ is

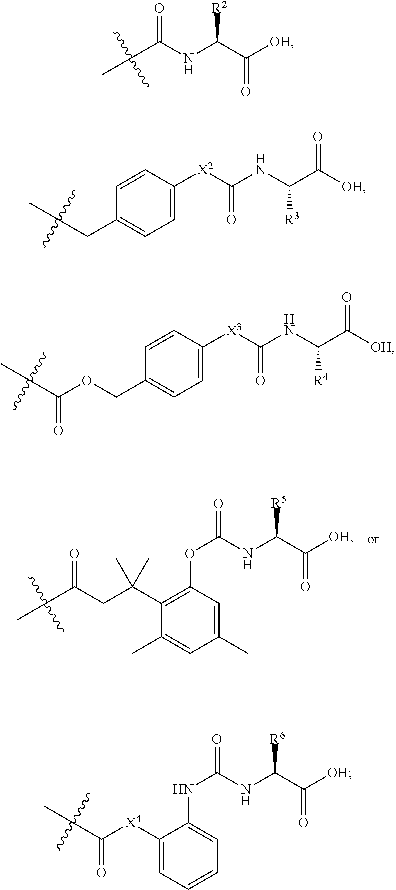

X² and X³ are each independently O or NH; X⁴ is O, NH, CH₂, or C(Me)₂; and R², R³, R⁴, R⁵, and R⁶ are each independently —(CH₂)₃—NH(NH)—NH₂, —(CH₂)₃—NH₂, —(CH₂)₄—NH₂, —CH₂—C(O)OH, —(CH₂)₂—C(O)OH,

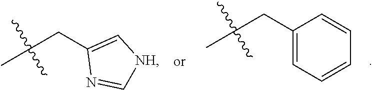 [SMILES: *Cc1c[nH]cn1 ; *Cc1ccccc1]

### BRIEF DESCRIPTION OF THE DRAWINGS

FIG. 1 illustrates that recombinant Pseudomonas sp. CPG2 produced and purified from E. coli cell lysates migrates as a single band at ˜42 kDa by SDS-PAGE/coomassie, according to the working examples.

FIG. 2 provides the results of incubating APdMG-Glu, an embodiment of a prodrug compound of the present technology, with increasing concentration of recombinant CPG2 where glutamate release was measured in an Amplex Red assay, according to the working examples.

FIG. 3 provides the results of HCC827 cells incubated for 48 hrs. with or without CPG2 in the presence of APdMG-Glu where cell viability was measured by Cell-Titre Glo, according to the working examples. The viability of the cells incubated with the active version of the drug (O-(3-aminopropyl)-des-morpholino-gefitinib) are shown for comparison.

FIG. 4 provides IC₅₀ and selectivity data for APdMG-Glu with HCC827 cells, according to the working examples.

FIG. 5 provides IC₅₀ and selectivity data for APdMG-Glu with PC9 cells, according to the working examples.

### DETAILED DESCRIPTION OF THE INVENTION

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### Definitions

Unless defined otherwise, all technical and scientific terms used herein have the meaning commonly understood by a person skilled in the art to which this disclosure belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, or B and C.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S¹⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; and nitriles (i.e., CN).

Substituted ring groups such as substituted cycloalkyl, aryl, heterocyclyl, and heteroaryl groups also include rings and ring systems in which a bond to a hydrogen atom is replaced with a bond to a carbon atom. Therefore, substituted cycloalkyl, aryl, heterocyclyl, and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi-, or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl, or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two, or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic, and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl, or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3, or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated, and saturated ring systems, such as, for example, imidazolyl, and imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo, or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl, or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are heteroarylene groups, and so forth. Substituted groups having a single point of attachment to the compound of the present technology are not referred to using the “ene” designation. Thus, e.g., chloroethyl is not referred to herein as chloroethylene.

Pharmaceutically acceptable salts of compounds described herein include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

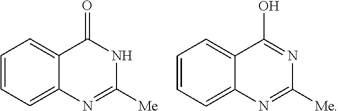

As another example, guanidines may exhibit the following isomeric forms in protic organic solution, also referred to as tautomers of each other:

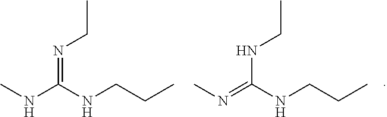

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

As used herein, the term “administration” of an agent to a subject includes any route of introducing or delivering the agent to a subject to perform its intended function. Administration can be carried out by any suitable route, including, but not limited to, intravenously, intramuscularly, intratumorally, intraperitoneally, subcutaneously, and other suitable routes as described herein. Administration includes self-administration and the administration by another.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. The terms encompass amino acid chains of any length, including full length proteins, wherein the amino acid residues are linked by covalent peptide bonds.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, or 10 of amino acid sequences.

As used herein the term “immune cell” refers to any cell that plays a role in the immune response. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes.

As used herein, the term “native immune cell” refers to an immune cell that naturally occurs in the immune system.

As used herein, the term “engineered immune cell” refers to an immune cell that is genetically modified.

The term “lymphocyte” refers to all immature, mature, undifferentiated, and differentiated white lymphocyte populations including tissue specific and specialized varieties. It encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, B-2 cells, and anergic AN1/T3 cell populations.

As used herein, the term “T-cell” includes naïve T cells, CD4+ T cells, CD8+ T cells, memory T cells, activated T cells, anergic T cells, tolerant T cells, chimeric B cells, and antigen-specific T cells.

As used herein “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL), TCR (i.e. heterologous T-cell receptor) modified lymphocytes and CAR (i.e. chimeric antigen receptor) modified lymphocytes. In another embodiment, the adoptive cell therapeutic composition comprises a cell type selected from a group consisting of T-cells, CD8+ cells, CD4+ cells, NK-cells, delta-gamma T-cells, regulatory T-cells and peripheral blood mononuclear cells. In another embodiment, TILs, T-cells, CD8+ cells, CD4+ cells, NK-cells, delta-gamma T-cells, regulatory T-cells or peripheral blood mononuclear cells form the adoptive cell therapeutic composition. In one embodiment, the adoptive cell therapeutic composition comprises T cells.

As used herein “tumor-infiltrating lymphocytes” or TILs refer to white blood cells that have left the bloodstream and migrated into a tumor.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments that lack the Fc fragment of intact antibody, clear more rapidly from the circulation, and may have less non-specific tissue binding of an intact antibody (Wahl et al., J. Nucl. Med. 24:316-325 (1983)). The antibodies of the invention comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), V_{NAR} fragments, Bi-specific T-cell engager (BiTE) antibodies, minibodies, disulfide-linked Fvs (sdFv), and anti-idiotypic (anti-Id) antibodies, intrabodies, fusion polypeptides, unconventional antibodies and antigen-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA, and IgY), class (e.g., IgG1, IgG2, IgG3, IgG4, IgA1, and IgA2), or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CHI, CH₂, and CH₃. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system. As used herein interchangeably, the terms “antigen-binding portion”, “antigen-binding fragment”, or “antigen-binding region” of an antibody, refer to the region or portion of an antibody that binds to the antigen and which confers antigen specificity to the antibody; fragments of antigen-binding proteins, for example, antibodies includes one or more fragments of an antibody that retain the ability to specifically bind to an antigen (e.g., an peptide/HLA complex). It has been shown that the antigen-binding function of an antibody can be performed by fragments of a full-length antibody. Examples of antigen-binding portions encompassed within the term “antibody fragments” of an antibody include a Fab fragment, a monovalent fragment consisting of the V_{L}, V_{H}, C_{L} and CHI domains; a F(ab)₂ fragment, a bivalent fragment comprising two Fab fragments linked by a disulfide bridge at the hinge region; a Fd fragment consisting of the V_{H} and CHI domains; a Fv fragment consisting of the V_{L} and V_{H} domains of a single arm of an antibody; a dAb fragment (Ward et al., Nature 341: 544-546 (1989)), which consists of a V_{H} domain; and an isolated complementarity determining region (CDR).

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, and urodele amphibians). The antibodies and antibody fragments can be produced in animals or produced outside of animals, such as from yeast or phage (e.g., as a single antibody or antibody fragment or as part of an antibody library).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science 242:423-426 (1988); and Huston et al., Proc. Natl. Acad. Sci. 85: 5879-5883 (1988). These antibody fragments are obtained using conventional techniques known to those of ordinary skill in the art, and the fragments are screened for utility in the same manner as are intact antibodies.

An “isolated antibody” or “isolated antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. In certain embodiments, the linker comprises amino acids having the sequence set forth in SEQ ID NO: 18 as provided below.

(SEQ ID NO: 18)
GGGGSGGGGSGGGGS

In certain embodiments, the nucleic acid sequence encoding the amino acid sequence of SEQ ID NO: 18 is set forth in SEQ ID NO: 19, which is provided below:

(SEQ ID NO: 19)
ggcggcggcggatctggaggtggtggctcaggtggcggaggctcc

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 27(6):455-51 (2008); Peter et al., J Cachexia Sarcopenia Muscle (2012); Shieh et al., J Imunol 183(4):2277-85 (2009); Giomarelli et al., Thromb Haemost 97(6):955-63 (2007); Fife et al., J Clin Invst 116(8):2252-61 (2006); Brocks et al., Immunotechnology 3(3): 173-84 (1997); Moosmayer et al., Ther Immunol 2(10):31-40 (1995) Agonistic scFvs having stimulatory activity have been described (see, e.g., Peter et al., J Biol Chem 25278(38):36740-7 (2003); Xie et al., Nat Biotech 15(8):768-71 (1997); Ledbetter et al., Crit Rev Immunol 17(5-6):427-55 (1997); Ho et al., Bio Chim Biophys Acta 1638(3):257-66 (2003)).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab¹) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. Sequences of Proteins of Immunological Interest, Fifth Edition, U.S. Department of Health and Human Services, NIH Publication No. 91-3242(1991)).

As used herein, the term “affinity” is meant a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes (e.g., either monovalent or multivalent). Methods for calculating the affinity of an antibody for an antigen are known in the art, comprising use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay). Nucleic acid molecules useful in the presently disclosed subject matter include any nucleic acid molecule that encodes a polypeptide or a fragment thereof. In certain embodiments, nucleic acid molecules useful in the presently disclosed subject matter include nucleic acid molecules that encode an antibody or an antigen-binding portion thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, Methods Enzymol. 152:399 (1987); Kimmel, A. R. Methods Enzymol. 152:507 (1987)).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% w/v formamide, and more preferably at least about 50% w/v formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% w/v SDS. In certain embodiments, hybridization will occur at 37° C. in 500 mM NaCl, 50 mM trisodium citrate, 1% w/v SDS, 35% w/v formamide, and 100 μg/ml denatured salmon sperm DNA (ssDNA). In certain embodiments, hybridization will occur at 42° C. in 250 mM NaCl, 25 mM trisodium citrate, 1% w/v SDS, 50% w/v formamide, and 200 μg ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In certain embodiments, wash steps will occur at 25° C. in 30 mM NaCl, 3 mM trisodium citrate, and 0.1% w/v SDS. In certain embodiments, wash steps will occur at 42° C. in 15 mM NaCl, 1.5 mM trisodium citrate, and 0.1% w/v SDS. In certain embodiments, wash steps will occur at 68° C. in 15 mM NaCl, 1.5 mM trisodium citrate, and 0.1% w/v SDS. Additional variations on these conditions will be readily apparent to those skilled in the art. Hybridization techniques are well known to those skilled in the art and are described, for example, in Benton and Davis (Science 196: 180 (1977)); Grunstein and Rogness (Proc. Natl. Acad. Sci., USA 72:3961 (1975)); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The terms “substantially homologous” or “substantially identical” mean a polypeptide or nucleic acid molecule that exhibits at least 50% or greater homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or about 99% homologous or identical at the amino acid level or nucleic acid to the sequence used for comparison (e.g., a wild-type, or native, sequence). In some embodiments, a substantially homologous or substantially identical polypeptide contains one or more amino acid amino acid substitutions, insertions, or deletions relative to the sequence used for comparison. In some embodiments, a substantially homologous or substantially identical polypeptide contains one or more non-natural amino acids or amino acid analogs, including, D-amino acids and retroinverso amino, to replace homologous sequences.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4: 1 1-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “analog” refers to a structurally related polypeptide or nucleic acid molecule having the function of a reference polypeptide or nucleic acid molecule.

As used herein, the term “a conservative sequence modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions, and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine; negatively-charged amino acids include aspartic acid and glutamic acid; and neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “ligand” refers to a molecule that binds to a receptor. In particular, the ligand binds a receptor on another cell, allowing for cell-to-cell recognition and/or interaction.

As used herein, the term, “co-stimulatory signaling domain,” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28 and 4-1BB, other costimulatory domains are contemplated for use with the CARs described herein. The inclusion of one or more co-stimulatory signaling domains can enhance the efficacy and expansion of T cells expressing CAR receptors. The intracellular signaling and co-stimulatory signaling domains can be linked in any order in tandem to the carboxyl terminus of the transmembrane domain.

As used herein, the term “chimeric co-stimulatory receptor” or “CCR” refers to a chimeric receptor that binds to an antigen and provides co-stimulatory signals, but does not provide a T-cell activation signal.

As used herein, “regulatory region” of a nucleic acid molecule means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, “operably linked” with reference to nucleic acid sequences, regions, elements or domains means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, production by recombinant means by using recombinant DNA methods means the use of the well-known methods of molecular biology for expressing proteins encoded by cloned DNA.

As used herein, a “host cell” is a cell that is used in to receive, maintain, reproduce and amplify a vector. A host cell also can be used to express the polypeptide encoded by the vector. The nucleic acid contained in the vector is replicated when the host cell divides, thereby amplifying the nucleic acids.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, a vector also includes “virus vectors” or “viral vectors.” Viral vectors are engineered viruses that are operatively linked to exogenous genes to transfer (as vehicles or shuttles) the exogenous genes into cells.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “disease” refers to any condition or disorder that damages or interferes with the normal function of a cell, tissue, or organ. Examples of diseases include neoplasia or pathogen infection of cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasia), or otherwise reduce the pathological consequences of the disease (e.g., a neoplasia). The effective amount is generally determined by the physician on a case-by-case basis and is within the skill of one in the art. Several factors are typically taken into account when determining an appropriate dosage to achieve an effective amount. These factors include general health, age, sex, body weight, and tolerance to drugs of the subject, the condition being treated, the degree, type and severity of the condition and the form and effective concentration of the engineered immune cells administered.

As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “secreted” is meant a polypeptide that is released from a cell via the secretory pathway through the endoplasmic reticulum, Golgi apparatus, and as a vesicle that transiently fuses at the cell plasma membrane, releasing the proteins outside of the cell. Small molecules, such as drugs, can also be secreted by diffusion through the membrane to the outside of cell.

As used herein, the term “specifically binds” or “specifically binds to” or “specifically target” is meant a polypeptide or fragment thereof that recognizes and binds a biological molecule of interest (e.g., a polypeptide), but which does not substantially recognize and bind other molecules in a sample, for example, a biological sample, which includes or expresses a tumor antigen.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

As used herein, the term “subject” refers to any animal (e.g., a mammal), including, but not limited to, humans, non-human primates, rodents, and the like (e.g., which is to be the recipient of a particular treatment, or from whom cells are harvested).

### Overview

Adoptive transfer of chimeric antigen receptor (CAR) T cells has been shown to be an effective therapy for B-cell ALL and other hematopoietic cancers. However, primary failures, incomplete remissions, and relapse still occurs via multiple mechanisms including loss of the target antigen. Solid tumors have been more resistant to this form of therapy. Therefore, greater potency and mechanisms to defeat the immunosuppressive tumor microenvironment are needed for many cancer types. Resistance of tumors to the activity of CAR T cells, antigen loss variant escape, and relapse are still important hurdles to the success of CAR T cell therapy.

In attending to these hurdles, the present inventors provided compositions and methods for adoptive cell therapy comprising engineered immune cells that express a prodrug converting enzyme and a receptor that binds to a target antigen, as disclosed in Int'l Appl. No. PCT/US2018/040629 (published as WO2019/006464), Int'l Appl. No. PCT/US2018/040633 (published as WO2018/040633), Int'l Appl. No. PCT/US2018/040640 (published as WO2019/006468) and Int'l Appl. No. PCT/US2018/040639 (published as WO 2019/006467), the disclosures of each of which are incorporated herein by reference (collectively referred to hereafter as the “SEAKER disclosures”). In further attending to these needs, the inventors of the present technology provide prodrug compounds (as well as pharmaceutical compositions thereof) useful with such compositions and in such methods.

In particular, the present technology includes prodrugs that provides active compounds similar to gefitinib. Gefitinib is a selective inhibitor of epidermal growth factor receptor's (EGFR) tyrosine kinase domain. The target protein (EGFR) is a member of a family of receptors (ErbB) which includes Her1(EGFR), Her2(erb-B2), Her3(erb-B3) and Her4 (Erb-B4). EGFR is overexpressed in the cells of certain types of human carcinomas—for example in lung cancers (e.g., non small cell lung cancer) and breast cancers. This leads to inappropriate activation of the anti-apoptotic Ras signalling cascade, eventually leading to uncontrolled cell proliferation. Research on gefitinib-sensitive non-small cell lung cancers has shown that a mutation in the EGFR tyrosine kinase domain is responsible for activating anti-apoptotic pathways. These mutations tend to confer increased sensitivity to tyrosine kinase inhibitors such as gefitinib and erlotinib. Of the types of non-small cell lung cancer histologies, adenocarcinoma is the type that most often harbors these mutations. Because of this, the prodrugs of the present technology include particular utility in treating such conditions via the compositions and methods provided in the SEAKER disclosures.

### Prodrug Compounds of the Present Technology

Thus, in an aspect, a prodrug compound (hereinafter alternatively referred to as “compound,” “compound(s) of the present technology,” or the like) of Formula I is provided

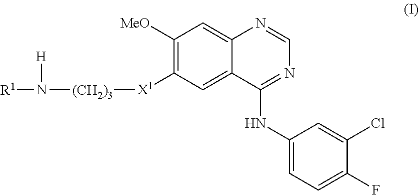

or a pharmaceutically acceptable salt, and/or solvate thereof, where X¹ is O or NH, R¹ is

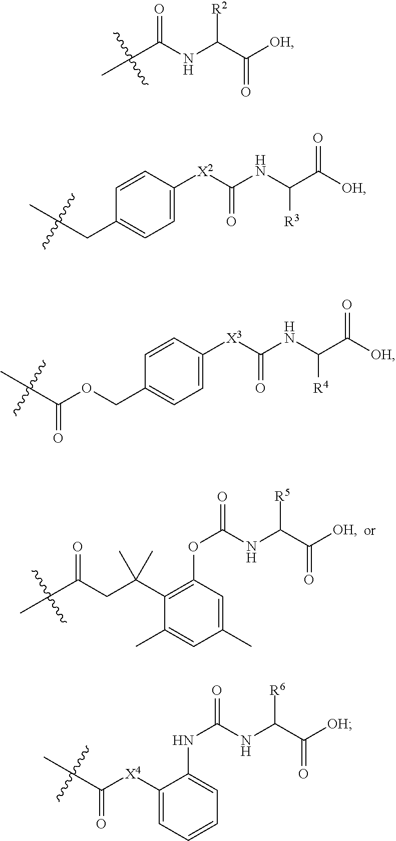

X² and X³ are each independently O or NH; X⁴ is O, NH, CH₂, or C(Me)₂; and R², R³, R⁴, R⁵, and R⁶ are each independently H, —(CH₂)₃—NH(NH)—NH₂, —(CH₂)₃—NH₂, —(CH₂)₄—NH₂, —CH₂—C(O)OH, —(CH₂)₂—C(O)OH,

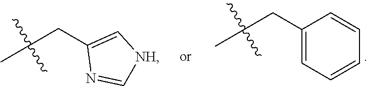 [SMILES: *Cc1c[nH]cn1 ; *Cc1ccccc1]

In any embodiment herein, it may be R¹ is

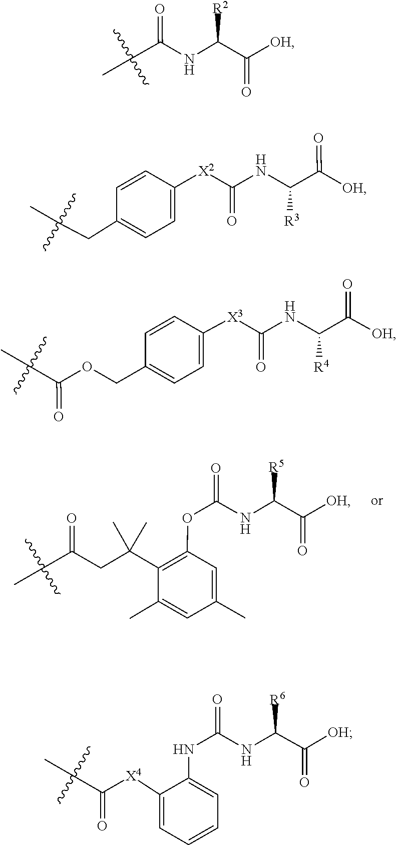

X² and X³ are each independently O or NH; X⁴ is O, NH, CH₂, or C(Me)₂; and R², R³, R⁴, R⁵, and R⁶ are each independently —(CH₂)₃—NH(NH)—NH₂, —(CH₂)₃—NH₂, —(CH₂)₄—NH₂, —CH₂—C(O)OH, —(CH₂)₂—C(O)OH,

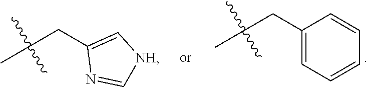 [SMILES: *Cc1c[nH]cn1 ; *Cc1ccccc1]

The prodrug compounds taught herein can be employed in any method or use requiring a prodrug compound. For example, any clinical application that employs an enzyme that can cleave the prodrugs provided herein to the active drug can use the provided prodrug compounds. Such methods and uses are not limited to the methods and uses described herein. For example, the prodrug compounds taught herein are not limited to use with the engineered immune cells (e.g., SEAKER cells) and related technologies as provided in the SEAKER disclosures. In some embodiments, the prodrug compounds provided herein are converted into an active drug by any suitable prodrug converting enzyme intracellularly (e.g. a suitable prodrug converting enzyme transduced into a cell or expressed by a cell). In some embodiments, the prodrug compounds provided herein are converted into an active drug by any suitable prodrug converting enzyme extracellularly. In some embodiments, the prodrug compounds are converted into an active drug by any suitable prodrug converting enzyme conjugated to ligand. In some embodiments, the ligand is an antibody. In some embodiments, the prodrug compounds are employed in combination with a directed enzyme prodrug therapy (DEPT). In some embodiments, the directed enzyme prodrug therapy is an antibody-directed enzyme prodrug therapy (ADEPT), a gene-directed enzyme prodrug therapy (GDEPT), a virus-directed enzyme prodrug therapy (VDEPT), a lectin-directed Enzyme-Activated Prodrug Therapy (LEAPT), polymer-directed enzyme prodrug therapy (PDEPT), clostridia-directed enzyme prodrug therapy (CDEPT) or any combination thereof.

In an aspect of the present technology, a composition is provided that includes any one of the herein-described embodiments of compounds of Formula I and a pharmaceutically acceptable carrier or one or more excipients or fillers (collectively referred to as “pharmaceutically acceptable carrier” unless otherwise specified). In a related aspect, a pharmaceutical composition is provided, the pharmaceutical composition including an effective amount of the compound of any one of the aspects and embodiments of compounds of Formula I for treating cancer in a subject; and a pharmaceutically acceptable carrier. Thus, the instant present technology provides pharmaceutical compositions and medicaments comprising any of the compounds disclosed herein (e.g., compounds of Formula I) and a pharmaceutically acceptable carrier or one or more excipients or fillers (collectively referred to as “pharmaceutically acceptable carrier” unless otherwise specified). The compositions may be used in the methods and treatments described herein and/or as described in the SEAKER disclosures. Such compositions and medicaments may include a therapeutically effective amount of any compound as described herein, including but not limited to a compound of Formula I. The pharmaceutical composition may be packaged in unit dosage form. The unit dosage form is effective in treating cancer, e.g., by reducing the size of a tumor associated with the cancer, when administered to a subject in need thereof.

The pharmaceutical compositions and medicaments may be prepared by mixing one or more compounds of the present technology, pharmaceutically acceptable salts thereof, or solvates thereof, with pharmaceutically acceptable carriers, excipients, binders, diluents or the like. The compounds and compositions described herein may be used to prepare formulations and medicaments that prevent or treat cancer. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts and/or solvates thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts (such as, e.g., described in the SEAKER disclosures) until, for example, a decrease in tumor size is observed. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the stage of the particular cancer, and the pharmacological activity of the compound being used. The determination of optimum dosages for a particular patient is well known to those skilled in the art.

Various assays and model systems can be readily employed to determine the therapeutic effectiveness of the treatment according to the present technology. Effectiveness of the compositions and methods of the present technology may also be demonstrated by a decrease in, e.g., the rate of growth of a tumor.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The compounds of the present technology can also be administered to a patient along with other conventional therapeutic agents that may be useful in the treatment of cancer. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially or synergistically be effective for the treatment of cancer.

A compound of the present technology may be administered to a patient in an amount or dosage suitable for therapeutic use or at doses that are nontoxic or tolerable to the patient. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg. In some embodiments, a compound of the present technology is administered at an amount that is about 10 to about 1000 times higher than that possible for the active drug. A suitable dose of a compound of the present technology can be from about 0.1 to 200 mg/Kg, such as about from 10 to 100 mg/Kg per patient per day or from 5 to 2000 mg/m² (e.g., 200 mg/m²).

### Prodrug Converting Enzymes

The engineered immune cells provided herein express at least one prodrug converting enzyme that converts the prodrug compound of the present technology into an active drug. In some embodiments, the prodrug converting enzyme is expressed on the surface of the engineered immune cell. In some embodiments, the prodrug converting enzyme is secreted by the engineered immune cell. The enzyme can be any enzyme which is capable of converting a prodrug into an active drug and which is not normally expressed on the surface of a cell (e.g., a mammalian cell, such as a human cell) or released into the circulation.

In some embodiments, the prodrug converting enzyme is a non-mammalian enzyme. Suitable non-mammalian enzymes include bacterial enzymes. Bacterial enzymes include carboxypeptidases, such as carboxypeptidase G2 (CPG2), which is a bacterial hydrolase enzyme isolated from Pseudomonas sp. RS-16 based on its ability to cleave N-linked glutamate from folate derivatives (Pseudomonas γ-glutamylhydrolase EC3.4.22.12, as disclosed in WO88/07378 and Levy and Goldstein, J. Biol. Chem. 242:2933 (1967)). CPG2 is specific for cleavage between an aromatic N-acyl moiety and glutamate, and has been used extensively in antibody-directed enzyme prodrug therapy (ADEPT) strategies, including several systems that advanced to human clinical trials for cancer.

In certain embodiments, the prodrug converting enzyme is CPG2. In some embodiments, CPG2 comprises the sequence set forth in SEQ ID NO: 1, which is a secreted form of CPG2 including the leader sequence for secretion.

(SEQ ID NO: 1)
MRPSIHRTAI AAVLATAFVA GTALAQKRDN VLFQAATDEQ
PAVIKTLEKL VNIETGTGDA EGIAAAGNFL EAELKNLGFT
VTRSKSAGLV VGDNIVGKIK GRGGKNLLLM SHMDTVYLKG
ILAKAPFRVE GDKAYGPGIA DDKGGNAVIL HTLKLLKEYG
VRDYGTITVL FNTDEEKGSF GSRDLIQEEA KLADYVLSFE
PTSAGDEKLS LGTSGIAYVQ VNITGKASHA GAAPELGVNA
LVEASDLVLR TMNIDDKAKN LRFNWTIAKA GNVSNIIPAS
ATLNADVRYA RNEDFDAAMK TLEERAQQKK LPEADVKVIV
TRGRPAFNAG EGGKKLVDKA VAYYKEAGGT LGVEERTGGG
TDAAYAALSG KPVIESLGLP GFGYHSDKAE YVDISAIPRR
LYMAARLIMD LGAGK

In some embodiments, CPG2 comprises the sequence set forth in SEQ ID NO: 2, which is a CPG2 polypeptide without the leader sequence of SEQ ID NO:1 and optimized for human expression.

(SEQ ID NO: 2)
ALAQKRDNVL FQAATDEQPA VIKTLEKLVN IETGTGDAEG
IAAAGNFLEA ELKNLGFTVT RSKSAGLVVG DNIVGKIKGR
GGKNLLLMSH MDTVYLKGIL AKAPFRVEGD KAYGPGIADD
KGGNAVILHT LKLLKEYGVR DYGTITVLFN TDEEKGSFGS
RDLIQEEAKL ADYVLSFEPT SAGDEKLSLG TSGIAYVQVQ
ITGKASHAGA APELGVNALV EASDLVLRTM NIDDKAKNLR
FQWTIAKAGQ VSNIIPASAT LNADVRYARN EDFDAAMKTL
EERAQQKKLP EADVKVIVTR GRPAFNAGEG GKKLVDKAVA
YYKEAGGTLG VEERTGGGTD AAYAALSGKP VIESLGLPGF
GYHSDKAEYV DISAIPRRLY MAARLIMDLG AGK

In some embodiments, CPG2 comprises the sequence set forth in SEQ ID NO: 3, which is an exemplary secreted form of CPG2 including the leader sequence for secretion and optimized for human expression.

(SEQ ID NO: 3)
MRPSIHRTAI AAVLATAFVA GTALAQKRDN VLFQAATDEQ
PAVIKTLEKL VNIETGTGDA EGIAAAGNFL EAELKNLGFT
VTRSKSAGLV VGDNIVGKIK GRGGKNLLLM SHMDTVYLKG
ILAKAPFRVE GDKAYGPGIA DDKGGNAVIL HTLKLLKEYG
VRDYGTITVL FNTDEEKGSF GSRDLIQEEA KLADYVLSFE
PTSAGDEKLS LGTSGIAYVQ VQITGKASHA GAAPELGVNA
LVEASDLVLR TMNIDDKAKN LRFQWTIAKA GQVSNIIPAS
ATLNADVRYA RNEDFDAAMK TLEERAQQKK LPEADVKVIV
TRGRPAFNAG EGGKKLVDKA VAYYKEAGGT LGVEERTGGG
TDAAYAALSG KPVIESLGLP GFGYHSDKAE YVDISAIPRR
LYMAARLIMD LGAGK

In some embodiments, CPG2 comprises the sequence set forth in SEQ ID NOS: 6 or 7 below, which are exemplary transmembrane forms of CPG2 optimized for human expression with a CD8 leader sequence (e.g., MALPVTALLLPLALLLHAARP (SEQ ID NO: 4)), a transmembrane portion and optionally a CD8 intracellular portion (e.g., LYCNHRNRRRVCKCPRPVVKSGDKPSLSARYV (SEQ ID NO: 17) or LYCNHRNRRRVGGGRPVVKSGDKPSLSARYV (SEQ ID NO: 5; with a disrupted Lyk binding domain)):

(SEQ ID NO: 6)
MALPVTALLL PLALLLHAAR PALAQKRDNV LFQAATDEQP
AVIKTLEKLV NIETGTGDAE GIAAAGNFLE AELKNLGFTV
TRSKSAGLVV GDNIVGKIKG RGGKNLLLMS HMDTVYLKGI
LAKAPFRVEG DKAYGPGIAD DKGGNAVILH TLKLLKEYGV
RDYGTITVLF NTDEEKGSFG SRDLIQEEA KLADYVLSFEP
TSAGDEKLSL GTSGIAYVQV QITGKASHAG AAPELGVNAL
VEASDLVLRT MNIDDKAKNL RFQWTIAKAG QVSNIIPASA
TLNADVRYAR NEDFDAAMKT LEERAQQKKL PEADVKVIVT
RGRPAFNAGE GGKKLVDKAV AYYKEAGGTL GVEERTGGGT
DAAYAALSGK PVIESLGLPG FGYHSDKAEY VDISAIPRRL
YMAARLIMDL GAGKYPYDVP DYAGGGLYCN HRNRRRVCKC
PRPVVKSGDK PSLSARYV
(SEQ ID NO: 7)
MALPVTALLLPLALLLHAARPALAQKRDNVLFQAATDEQPAVI
KTLEKLVNIETGTGDAEGIAAAGNFLEAELKNLGFTVTRSKSA
GLVVGDNIVGKIKGRGGKNLLLMSHMDTVYLKGILAKAPFRVE
GDKAYGPGIADDKGGNAVILHTLKLLKEYGVRDYGTITVLFNT
DEEKGSFGSRDLIQEEAKLADYVLSFEPTSAGDEKLSLGTSGI
AYVQVQITGKASHAGAAPELGVNALVEASDLVLRTMNIDDKAK
NLRFQWTIAKAGQVSNIIPASATLNADVRYARNEDFDAAMKTL
EERAQQKKLPEADVKVIVTRGRPAFNAGEGGKKLVDKAVAYYK
EAGGTLGVEERTGGGTDAAYAALSGKPVIESLGLPGFGYHSDK
AEYVDISAIPRRLYMAARLIMDLGAGKYPYDVPDYAGGGIYIW
APLAGTCGVLLLSLVITLYCNHRNRRRVGGGRPVVKSGDKPSL
SARYV.

In some embodiments, the prodrug converting enzyme is a β-lactamase, such as an Enterobacter cloacae β-lactamase. β-Lactamase (EC 3.5.2.6) is a serine protease produced by various bacteria; it catalyzes the hydrolysis of the β-lactam moiety in penicillin and other similar β-lactam antibiotics to 3-amino acid. β-lactamase is highly selective to the β-lactam containing compounds. Cephalosporin has been widely utilized as the core of β-lactamase-activated prodrugs due to its ability of releasing the drug from C3′ position following the cleavage of β-lactam ring by β-lactamase.

In some embodiments, β-Lactamase comprises the sequence set forth in SEQ ID NO: 8, which is a secreted form of β-Lactamase including the leader sequence for secretion.

(SEQ ID NO: 8)
MMRKSLCCAL LLGISCSALA TPVSEKQLAE VVANTITPLM
KAQSVPGMAV AVIYQGKPHY YTFGKADIAA NKPVTPQTLF
ELGSISKTFT GVLGGDAIAR GEISLDDAVT RYWPQLTGKQ
WQGIRMLDLA TYTAGGLPLQ VPDEVTDNAS LLRFYQNWQP
QWKPGTTRLY ANASIGLFGA LAVKPSGMP YEQAMTTRVLK
PLKLDHTWIN VPKAEEAHYA WGYRDGKAVR VSPGMLDAQA
YGVKTNVQDM ANWVMANMAP ENVADASLKQ GIALAQSRYW
RIGSMYQGLG WEMLNWPVEA NTVVEGSDSK VALAPLPVAE
VNPPAPPVKA SWVHKTGSTG GFGSYVAFIP EKQIGIVMLA
NTSYPNPARV EAAYHILEAL Q

In some embodiments, β-Lactamase comprises the sequence set forth in SEQ ID NO: 9, which is a β-Lactamase polypeptide without the leader sequence of SEQ ID NO: 8 and optimized for human expression.

(SEQ ID NO: 9)
TPVSEKQLAE VVANTITPLM AAQSVPGMAV AVIYQGKPHY
YTFGKADIAA NKPVTPQTLF ELGSISKTFT GVLGGDAIAR
GEISLDDAVT RYWPQLTGKQ WQGIRMLDLA TYTAGGLPLQ
VPDEVTDNAS LLRFYQNWQP QWKPGTTRLY ANASIGLFGA
LAVKPSGMPY EQAMTTRVLK PLKLDHTWIN VPKAEEAHYA
WGYRDGKAVR VSPGMLDAQA YGVKTNVQDM ANWVMANMAP
ENVADASLKQ GIALAQSRYW RIGSMYQGLG WEMLNWPVEA
NTVVEGSDSK VALAPLPVAE VNPPAPPVKA SWVHKTGSTG
GFGAYVAFIP EKQIGIVMLA NTSYPNPARV EAAYHILEAL
Q

In some embodiments, β-Lactamase comprises the sequence set forth in SEQ ID NO: 10, which is an exemplary secreted form of β-Lactamase including the leader sequence for secretion and optimized for human expression.

(SEQ ID NO: 10)
MMRKSLCCAL LLGISCSALA TPVSEKQLAE VVANTITPLM
AAQSVPGMAV AVIYQGKPHY YTFGKADIAA NKPVTPQTLF
ELGSISKTFT GVLGGDAIAR GEISLDDAVT RYWPQLTGKQ
WQGIRMLDLA TYTAGGLPLQ VPDEVTDNAS LLRFYQNWQP
QWKPGTTRLY ANASIGLFGA LAVKPSGMPY EQAMTTRVLK
PLKLDHTWIN VPKAEEAHYA WGYRDGKAVR VSPGMLDAQA
YGVKTNVQDM ANWVMANMAP ENVADASLKQ GIALAQSRYW
RIGSMYQGLG WEMLNWPVEA NTVVEGSDSK VALAPLPVAE
VNPPAPPVKA SWVHKTGSTG GFGAYVAFIP EKQIGIVMLA
NTSYPNPARV EAAYHILEAL Q

In some embodiments, β-Lactamase comprises the sequence set forth in SEQ ID NO: 11, which is an exemplary transmembrane form of β-Lactamase optimized for human expression with a CD8 leader sequence (e.g., SEQ ID NO: 4), a transmembrane portion and optionally a CD8 intracellular portion with a disrupted Lyk binding domain (e.g., SEQ ID NO: 5).

(SEQ ID NO: 11)
MALPVTALLL PLALLLHAAR PTPVSEKQLA EVVANTITPL
MAAQSVPGMA VAVIYQGKPH YYTFGKADIA ANKPVTPQTL
FELGSISKTF TGVLGGDAIA RGEISLDDAV TRYWPQLTGK
QWQGIRMLDL ATYTAGGLPL QVPDEVTDNA SLLRFYQNWQ
PQWKPGTTRL YANASIGLFG ALAVKPSGMP YEQAMTTRVL
KPLKLDHTWI NVPKAEEAHY AWGYRDGKAV RVSPGMLDAQ
AYGVKTNVQD MANWVMANMA PENVADASLK QGIALAQSRY
WRIGSMYQGL GWEMLNWPVE ANTVVEGSDS KVALAPLPVA
EVNPPAPPVK ASWVHKTGST GGFGAYVAFI PEKQIGIVML
ANTSYPNPAR VEAAYHILEA LQYPYDVPDY A

In some embodiments, the transmembrane β-lactamase enzyme has the following amino acid sequence, which includes a signal peptide that is removed during processing in the endoplasmic reticulum:

(SEQ ID NO: 12)
MALPVTALLL PLALLLHAAR PTPVSEKQLA EVVANTITPL
MAAQSVPGMA VAVIYQGKPH YYTFGKADIA ANKPVTPQTL
FELGSISKTF TGVLGGDAIA RGEISLDDAV TRYWPQLTGK
QWQGIRMLDL ATYTAGGLPL QVPDEVTDNA SLLRFYQNWQ
PQWKPGTTRL YANASIGLFG ALAVKPSGMP YEQAMTTRVL
KPLKLDHTWI NVPKAEEAHY AWGYRDGKAV RVSPGMLDAQ
AYGVKTNVQD MANWVMANMA PENVADASLK QGIALAQSRY
WRIGSMYQGL GWEMLNWPVE ANTVVEGSDS KVALAPLPVA
EVNPPAPPVK ASWVHKTGST GGFGAYVAFI PEKQIGIVML
ANTSYPNPAR VEAAYHILEA LQYPYDVPDY AGGGLYCNHR
NRRRVCKCPR PVVKSGDKPS LSARYV

Examples of other suitable non-mammalian enzymes include nitroreductases, such as an E. coli nitroreductase as disclosed in WO93/08288, thymidine kinase (tk), including viral tk such as VZV or HSV tk, β-glucosidase, β-glucoronidase, penicillin V amidase, penicillin G amidase and cytosine deaminase.

In some embodiments, the enzyme is a mammalian enzyme which does not naturally occur in a human. In some embodiments, the enzyme is a human enzyme which is not normally accessible to the prodrug compound disclosed herein, is expressed in limited quantities outside of cells, is expressed in a compartment that is not reached by the prodrug compound disclosed herein, is expressed by a cell that can be killed by the activation of the prodrug compound disclosed herein but is not essential to the patient, and/or is expressed by a cell that is resistant to the active drug or not killed by the active drug. This includes, for example, enzymes from other species as well as mammalian enzymes which are altered in a manner which is selective for the prodrug compound. In some embodiments, the alteration means that the conversion of the prodrug compound to an active drug by the natural enzyme will be at a rate one or more orders of magnitude less than the rate at which the altered enzyme operates. Altered enzymes can be made by standard recombinant DNA techniques, e.g., by cloning the enzyme, determining its gene sequence and altering the gene sequence by methods such as site-directed mutagenesis. In some embodiments, the prodrug converting enzyme is carboxypeptidase A, or a mutant thereof, e.g. a T268G mutant of carboxypeptidase A.

For expression of the secreted prodrug converting enzymes or transmembrane prodrug converting enzymes, eukaryotic based expression systems (e.g., plasmid or viral-based systems, such as retroviral transduction) are employed. For secretion or transmembrane expression, a signal peptide is included at the N-terminus of protein. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the N-terminus of the prodrug converting enzyme (e.g., a CPG2 enzyme or β-Lactamase). In certain embodiments, the signal peptide comprises a CPG2 signal sequence or a β-Lactamase signal sequence.

In certain embodiments, the signal peptide comprises a CPG2 signal sequence comprising amino acids having the sequence set forth in SEQ ID NO: 13 as provided below.

(SEQ ID NO: 13)
MRPSIHRTAI AAVLATAFVA GT

The nucleotide sequence encoding the amino acid sequence of SEQ ID NO: 13 is set forth in SEQ ID NO: 14, which is provided below:

(SEQ ID NO: 14)
atgcgaccgagtatccacagaacagcaatagctgctgtg
cttgcaacagcgtttgtagcgggcacg

In certain embodiments, the signal peptide comprises a CD8 signal polypeptide comprising amino acids having the sequence set forth in SEQ ID NO: 4 as provided below.

(SEQ ID NO: 4)
MALPVTALLLPLALLLHAARP

The nucleotide sequence encoding the amino acid sequence of SEQ ID NO: 4 is set forth in SEQ ID NO: 15, which is provided below:

(SEQ ID NO: 15)
atggccctgccagtaacggctctgctgctgccacttgct
ctgctcctccatgcagccaggcct.

For cell surface expression of the prodrug converting enzyme, the expressed protein is anchored to the cell membrane. In some embodiments, the prodrug converting enzyme comprises a transmembrane domain. In some embodiments, the transmembrane domain is covalently attached to the C-terminus of the CPG2 enzyme or β-lactamase. In some embodiments, the transmembrane domain is covalently attached to the N-terminus of the CPG2 enzyme β-lactamase. In some embodiments, the transmembrane domain is CD8 transmembrane domain. In some embodiments, the transmembrane domain comprises a transmembrane spanning portion. In some embodiments, the membrane-spanning portion comprises the following sequence:

(SEQ ID NO: 16)
IYTWAPLAGTCGVLLLSLVIT

In some embodiments, the CD8 transmembrane domain also comprise an intracellular portion having the amino acid sequence:

(SEQ ID NO: 17)
LYCNHRNRRRVCKCPRPVVKSGDKPSLSARYV

In some embodiments, the CD8 transmembrane domain comprise an intracellular portion in which the natural Lyk binding domain is disrupted to uncouple the endogenous function of CD8 from the CPG2 or β-lactamase molecule. For example, in some embodiments, the CD8 transmembrane domain portion with disrupted Lyk binding domain has the following sequence: LYCNHRNRRRVGGGRPVVKSGDKPSLSARYV (SEQ ID NO: 5)

In some alternative embodiments, a construct for expression of a CPG2 enzyme comprises a CPG2 without a signal peptide.

In some alternative embodiments, a construct for expression of a β-lactamase enzyme comprises a β-lactamase without a signal peptide.

In some embodiments, the CPG2 enzyme or β-lactamase is attached to the cell surface via a glycosylphosphatidylinositol (GPI)-linker. Glypiated (GPI-linked) proteins contain a cleavable, hydrophobic amino-terminal signal sequence that targets the protein to the lumen of the endoplasmic reticulum (ER) and a cleavable, carboxy-terminal signal sequence that directs GPI anchoring. The GPI-anchoring signal consists of a hydrophobic region separated from the GPI-attachment site (ω-site) by a hydrophilic spacer region. (See e.g. Galian et al. (2012) J Biol Chem. 11; 287(20): 16399-16409).

Because the expressed secreted or transmembrane prodrug converting enzymes are processed through the Golgi apparatus and endoplasmic reticulum, they can become glycosylated, which may lead in a reduction in activity of the enzyme compared to its non-glycosylated form. Accordingly, in some embodiments, prodrug converting enzymes is altered from its native sequence by substitution, deletion or insertion at one or more (e.g., two, three or four) glycosylation sites. For example, within the primary amino acid sequence of CPG2, there are three such consensus glycosylation motifs, located at residues Asn 222, Asn 264 and Asn 272 of SEQ ID NO: 1. In some embodiments, one or more of these glycosylation sites is altered to remove the glycosylation site. In some embodiments, one or more of Asn 222, Asn 264 and Asn 272 is removed. In some embodiments, one or more of Asn 222, Asn 264 and Asn 272 is substituted with leucine or glutamine (see, e.g., SEQ ID NOS: 2, 3, 6, and 7 which represent exemplary CPG2 polypeptides for expression).

In some embodiments, at least 1, 2, 3, 4, 5, 6, 7, 8, 9, 10 or more glycosylation sites in the prodrug converting enzyme are substituted. In some embodiments, 1, 2, 3, 4, 5 or more amino acids are deleted or inserted at or near the consensus glycosylation site. Typically, the alteration will be such that the enzyme retains its ability to convert a prodrug compound to an active drug at substantially the same rate as the unchanged, non-glycosylated enzyme. In this context, “substantially unchanged,” is within 1 order of magnitude, such as from about 2-fold less activity to 2, 3, 4, 5, 6, 7, 8, 9, or 10-fold or more activity.

In some embodiments, the enzyme is altered by truncation, substitution, deletion or insertion relative to its native form so as long as the activity of the enzyme is substantially unchanged as defined above. For example, in some embodiments small truncations in the N- and/or C-terminal sequence (e.g., about 1 to about 20 amino acids) relative to the native full-length sequence are employed. In some embodiments, such truncations are needed to link the polypeptide to the various other signal sequences or peptides as described herein. The activity of the altered enzyme can be measured in suitable model systems which can be prepared in routine ways known in the art.

### Targeting Ligands and Target Antigens

In some embodiments, the engineered immune cells provided herein express a T-cell receptor (TCR) or other cell-surface ligand that binds to a target antigen, such as a tumor antigen. The cell-surface ligand can be any molecule that directs an immune cell to a target site (e.g., a tumor site). Exemplary cell surface ligands include, for example endogenous receptors, engineered receptors, or other specific ligands to achieve targeting of the immune cell to a target site. In some embodiments, the receptor is a T cell receptor. In some embodiments, the T cell receptor is a wild-type, or native, T-cell receptor that binds to a target antigen. In some embodiments, the receptor, e.g. a T cell receptor, is non-native receptor (e.g., not endogenous to the immune cells). In some embodiments, the receptor is a chimeric antigen receptor (CAR), for example, a T cell CAR, that binds to a target antigen.

In some embodiments, the target antigen expressed by a tumor cell. In some embodiments, the target antigen is expressed on the surface of a tumor cell. In some embodiments, the target antigen is a cell surface receptor. In some embodiments, the target antigen is a cell surface glycoprotein. In some embodiments, the target antigen is secreted by a tumor cell. In some embodiments, the target antigen is localized to the tumor microenvironment. In some embodiments, the target antigen is localized to the extracellular matrix or stroma of the tumor microenvironment. In some embodiments, the target antigen is expressed by one or more cells located within the extracellular matrix or stroma of the tumor microenvironment.

In some embodiments, the target antigen is a tumor antigen selected from among 5T4, alpha 5β1-integrin, 707-AP, A33, AFP, ART-4, B7H4, BAGE, Bcl-2, β-catenin, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1. In certain embodiments, the target antigen is a tumor antigen selected from among CD19, WT1, PRAME.

Without limiting the foregoing, exemplary cancers can be treated by targeting the associated provided antigens include: leukemia/lymphoma (CD19, CD20, CD22, ROR1, CD33); multiple myeloma (B-cell maturation antigen (BCMA)); prostate cancer (PSMA, WT1, Prostate Stem Cell antigen (PSCA), SV40 T); breast cancer (HER2, ERBB2); stem cell cancer (CD133); ovarian cancer (L1-CAM, extracellular domain of MUC16 (MUC-CD), folate binding protein (folate receptor), Lewis Y); renal cell carcinoma (carboxy-anhydrase-IX (CAIX); melanoma (GD2); and pancreatic cancer (mesothelin, CEA, CD24).

Typical therapeutic anti-cancer mAb, like those that bind to CD19, recognize cell surface proteins, which constitute only a tiny fraction of the cellular protein content. Most mutated or oncogenic tumor associated proteins are typically nuclear or cytoplasmic. In certain instances, these intracellular proteins can be degraded in the proteasome, processed and presented on the cell surface by MHC class I molecules as T cell epitopes that are recognized by T cell receptors (TCRs). The development of mAb that mimic TCR function, “TCR mimic (TCRm)” or “TCR-like”; (i.e., that recognize peptide antigens of key intracellular proteins in the context of MHC on the cell surface) greatly extends the potential repertoire of tumor targets addressable by potent mAb. TCRm Fab, or scFv, and mouse IgG specific for the melanoma Ags, NY-ESO-1, hTERT, MART 1, gp100, and PR1, among others, have been developed. The antigen binding portions of such antibodies can be incorporated into the CARs provided herein. HLA-A2 is the most common HLA haplotype in the USA and EU (about 40% of the population). Therefore, potent TCRm mAb and native TCRs against tumor antigens presented in the context of HLA-A2 are useful in the treatment of a large populations.

Accordingly, in some embodiments, target antigen is a tumor antigen presented in the context of an MHC molecule. In some embodiments, the MHC protein is a MHC class I protein. In some embodiments, the MHC Class I protein is an HLA-A, HLA-B, or HLA-C molecules. In some embodiments, target antigen is a tumor antigen presented in the context of an HLA-A2 molecule. mAbs for intracellular WT1 and PRAME antigens presented in the context of surface HLA-A2 molecules have previously been developed. IgG1, afucosylated Fc forms, bispecific, BiTE, and CAR T cell formats have been made that exhibit potent therapeutic activity in multiple preclinical animal models. Such antibodies or portion thereof can be employed as described herein for the recognition of target antigens present on the surface of a target cell (e.g., a tumor cell) in the context of an MHC molecule.

### Chimeric Antigen Receptors

In some embodiments, the engineered immune cells provided herein express at least one chimeric antigen receptor (CAR). CARs are engineered receptors, which graft or confer a specificity of interest onto an immune effector cell. For example, CARs can be used to graft the specificity of a monoclonal antibody onto an immune cell, such as a T cell. In some embodiments, transfer of the coding sequence of the CAR is facilitated by nucleic acid vector, such as a retroviral vector.

There are currently three generations of CARs. In some embodiments, the engineered immune cells provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4+ and CD8+ T cells through their CD3ζ chain signaling domain in a single fusion molecule, independent of HLA-mediated antigen presentation.

In some embodiments, the engineered immune cells provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (e.g., CD3ζ). Preclinical studies have indicated that “Second Generation” CARs can improve the antitumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL).

In some embodiments, the engineered immune cells provided herein express a “third generation” CAR. “Third generation” CARs comprise those that provide multiple co-stimulation (e.g., CD28 and 4-1BB) and activation (e.g., CD3ζ).

In accordance with the presently disclosed subject matter, the CARs of the engineered immune cells provided herein comprise an extracellular antigen-binding domain, a transmembrane domain and an intracellular domain.

Extracellular Antigen-Binding Domain of a CAR. In certain embodiments, the extracellular antigen-binding domain of a CAR specifically binds a tumor antigen. In certain embodiments, the extracellular antigen-binding domain is derived from a monoclonal antibody (mAb) that binds to a tumor antigen. In some embodiments, the extracellular antigen-binding domain comprises an scFv. In some embodiments, the extracellular antigen-binding domain comprises a Fab, which is optionally crosslinked. In a some embodiments, the extracellular binding domain comprises a F(ab)₂. In some embodiments, any of the foregoing molecules are comprised in a fusion protein with a heterologous sequence to form the extracellular antigen-binding domain. In certain embodiments, the extracellular antigen-binding domain comprises a human scFv that binds specifically to a tumor antigen. In certain embodiments, the scFv is identified by screening scFv phage library with tumor antigen-Fc fusion protein.

In certain embodiments, the extracellular antigen-binding domain of a presently disclosed CAR has a high binding specificity and high binding affinity to a tumor antigen. For example, in some embodiments, the extracellular antigen-binding domain of the CAR (embodied, for example, in a human scFv or an analog thereof) binds to a particular tumor antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, or about 1×10⁻⁹ M or less. In certain non-limiting embodiments, the K_{d} is from about 3×10⁻⁹ M or less. In certain non-limiting embodiments, the K_{d} is from about 3×10⁻⁹ to about 2×10⁻⁷.

Binding of the extracellular antigen-binding domain (embodiment, for example, in an scFv or an analog thereof) of a presently disclosed tumor antigen-targeted CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the tumor antigen-targeted CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the scFv of a presently disclosed tumor antigen-targeted CAR is labeled with GFP.

In some embodiments, the extracellular antigen-binding domain of the expressed CAR binds to a tumor antigen that is expressed by a tumor cell. In some embodiments, the extracellular antigen-binding domain of the expressed CAR binds to a tumor antigen that is expressed on the surface of a tumor cell. In some embodiments, the extracellular antigen-binding domain of the expressed CAR binds to a tumor antigen that is expressed on the surface of a tumor cell in combination with an MHC protein. In some embodiments, the MHC protein is a MHC class I protein. In some embodiments, the MHC Class I protein is an HLA-A, HLA-B, or HLA-C molecules. In some embodiments, the extracellular antigen-binding domain of the expressed CAR binds to a tumor antigen that is expressed on the surface of a tumor cell not in combination with an MHC protein.

In some embodiments, the extracellular antigen-binding domain of the expressed CAR binds to tumor antigen selected from among 5T4, alpha 5β1-integrin, 707-AP, A33, AFP, ART-4, B7H4, BAGE, Bcl-2, β-catenin, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1. In certain embodiments, the extracellular antigen-binding domain of the expressed CAR binds to tumor antigen selected from among CD19, WT1, PRAME. Exemplary extracellular antigen-binding domains and methods of generating such domains and associated CARs are described in, e.g., WO2016/191246, WO2017/023859, WO2015/188141, WO2015/070061, WO2012/135854, WO2014/055668, which are incorporated by reference in their entirety, including the sequence listings provided therein.

In some embodiments, the extracellular antigen-binding domain of the expressed CAR binds to a tumor antigen. In some embodiments, the extracellular antigen-binding domain of the expressed CAR binds to a tumor antigen presented in the context of an MHC molecule. In some embodiments, the extracellular antigen-binding domain of the expressed CAR binds to a tumor antigen presented in the context of an HLA-A2 molecule. Examples of suitable tumor antigens include, but are not limited to 5T4, alpha 5β1-integrin, 707-AP, A33, AFP, ART-4, B7H4, BAGE, Bcl-2, β-catenin, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1. In certain embodiments, the extracellular antigen-binding domain of the expressed CAR binds to tumor antigen selected from among CD19, WT1, PRAME.

In certain embodiments, the extracellular antigen-binding domain (e.g., human scFv) comprises a heavy chain variable (V_{H}) region and a light chain variable (V_{L}) region, optionally linked with a linker sequence, for example a linker peptide (e.g., SEQ ID NO: 18), between the heavy chain variable (V_{H}) region and the light chain variable (V_{L}) region. In certain embodiments, the extracellular antigen-binding domain is a human scFv-Fc fusion protein or full length human IgG with V_{H} and V_{L} regions.

In certain non-limiting embodiments, an extracellular antigen-binding domain of the presently disclosed CAR can comprise a linker connecting the heavy chain variable (V_{H}) region and light chain variable (V_{L}) region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in SEQ ID NO: 18. In certain embodiments, the nucleotide sequence encoding the amino acid sequence of SEQ ID NO: 18 is set forth in SEQ ID NO: 19.

In certain embodiments, the extracellular antigen-binding domain comprises a human scFv that binds to a CD19 antigen. In some embodiments, the scFv comprises a polypeptide having an amino acid sequence of SEQ ID NO: 20.

(SEQ ID NO: 20)
MALPVTALLLPLALLLHAEVKLQQSGAELVRPGSSVKIS
CKASGYAFSSYWMNWVKQRPGQGLEWIGQIYPGDGDTNY
NGKFKGQATLTADKSSSTAYMQLSGLTSEDSAVYFCARK
TISSVVDFYFDYWGQGTTVTVSSGGGGSGGGGSGGGGSD
IELTQSPKFMSTSVGDRVSVTCKASQNVGTNVAWYQQKP
GQSPKPLIYSATYRNSGVPDRFTGSGSGTDFTLTITNVQ
SKDLADYFCQQYNRYPYTSGGGTKLEIKR

In some embodiments, the scFv comprises a polypeptide having an amino acid sequence that is at least 80%, at least 85%, at least 90%, or at least 95% identical to SEQ ID NO: 20. For example, the scFv comprises a polypeptide having an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 20.

In some embodiments, the scFv is encoded by a nucleic acid having a nucleic acid sequence of SEQ ID NO: 21.

(SEQ ID NO: 21)
ATGGCTCTCCCAGTGACTGCCCTACTGCTTCCCCTAGCGC
TTCTCCTGCATGCAGAGGTGAAGCTGCAGCAGTCTGGGGC
TGAGCTGGTGAGGCCTGGGTCCTCAGTGAAGATTTCCTGC
AAGGCTTCTGGCTATGCATTCAGTAGCTACTGGATGAACT
GGGTGAAGCAGAGGCCTGGACAGGGTCTTGAGTGGATTGG
ACAGATTTATCCTGGAGATGGTGATACTAACTACAATGGA
AAGTTCAAGGGTCAAGCCACACTGACTGCAGACAAATCCT
CCAGCACAGCCTACATGCAGCTCAGCGGCCTAACATCTGA
GGACTCTGCGGTCTATTTCTGTGCAAGAAAGACCATTAGT
TCGGTAGTAGATTTCTACTTTGACTACTGGGGCCAAGGGA
CCACGGTCACCGTCTCCTCAGGTGGAGGTGGATCAGGTGG
AGGTGGATCTGGTGGAGGTGGATCTGACATTGAGCTCACC
CAGTCTCCAAAATTCATGTCCACATCAGTAGGAGACAGGG
TCAGCGTCACCTGCAAGGCCAGTCAGAATGTGGGTACTAA
TGTAGCCTGGTATCAACAGAAACCAGGACAATCTCCTAAA
CCACTGATTTACTCGGCAACCTACCGGAACAGTGGAGTCC
CTGATCGCTTCACAGGCAGTGGATCTGGGACAGATTTCAC
TCTCACCATCACTAACGTGCAGTCTAAAGACTTGGCAGAC
TATTTCTGTCAACAATATAACAGGTATCCGTACACGTCCG
GAGGGGGGACCAAGCTGGAGATCAAACGG

In some embodiments, the scFv is encoded by a nucleic acid having a nucleic acid sequence that is at least 80%, at least 85%, at least 90%, or at least 95% identical to SEQ ID NO: 21. In some embodiments, the scFv is encoded by a nucleic acid having a nucleic acid sequence of SEQ ID NO: 21. In some embodiments, the scFv is encoded by a nucleic acid having a nucleic acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 21.

Additionally or alternatively, in some embodiments, the extracellular antigen-binding domain can comprise a leader or a signal peptide sequence that directs the nascent protein into the endoplasmic reticulum. The signal peptide or leader can be essential if the CAR is to be glycosylated and anchored in the cell membrane. The signal sequence or leader sequence can be a peptide sequence (about 5, about 10, about 15, about 20, about 25, or about 30 amino acids long) present at the N-terminus of the newly synthesized proteins that direct their entry to the secretory pathway.

In certain embodiments, the signal peptide is covalently joined to the N-terminus of the extracellular antigen-binding domain. In certain embodiments, the signal peptide comprises a human CD8 signal polypeptide comprising amino acids having the sequence set forth in SEQ ID NO: 22 as provided below: MALPVTALLLPLALLLHAARP (SEQ ID NO: 22).

The nucleotide sequence encoding the amino acid sequence of SEQ ID NO: 22 is set forth in SEQ ID NO: 23, which is provided below:

(SEQ ID NO: 23)
ATGGCCCTGCCAGTAACGGCTCTGCTGCTGCCACTTGCTC
TGCTCCTCCATGCAGCCAGGCCT

In certain embodiments, the signal peptide comprises a human CD8 signal polypeptide comprising amino acids having the sequence set forth in SEQ ID NO: 24 as provided below: MALPVTALLLPLALLLHA (SEQ ID NO: 24).

The nucleotide sequence encoding the amino acid sequence of SEQ ID NO: 24 is set forth in SEQ ID NO: 25, which is provided below:

(SEQ ID NO: 25)
ATGGCTCTCCCAGTGACTGCCCTACTGCTTCCCCT
AGCGCTTCTCCTGCATGCA.

Transmembrane Domain of a CAR. In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (e.g., a transmembrane peptide not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a UniProtKB Reference No: P10747 or NCBI Reference No: NP006130 (SEQ ID NO: 26), or fragments thereof, and/or may optionally comprise up to one or up to two or up to three conservative amino acid substitutions. In certain embodiments, the CD28 polypeptide can have an amino acid sequence that is a consecutive portion of SEQ ID NO: 26 which is at least 20, or at least 30, or at least 40, or at least 50, and up to 220 amino acids in length. Additionally or alternatively, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 26. In certain embodiments, the CAR of the present disclosure comprises a transmembrane domain comprising a CD28 polypeptide, and optionally an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 26. In certain embodiments, the CD28 polypeptide comprised in the transmembrane domain has an amino acid sequence of amino acids 153 to 179 of SEQ ID NO: 26.

SEQ ID NO: 26 is provided below:

(SEQ ID NO: 26)
MLRLLLALNLFPSIQVTGNKILVKQSPMLVAYDNA
LSCKYSYNLFSREFRASLHKGLDSAVEVCWYGNY
SQQLQVYSKTGFNCDGKLGNESVTFYLQNLYQTDI
YFCKIEVMYPPPYLDNEKSNGTIIHVKGKHLCPSP
LFPGPSKPFWVLVWGGVLACYSLLVTVAFIIFWVR
SKRSRLLHSDYMNMTPRRPGPTRKHYQPYAPPRDF
AAYRS

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain (and optionally the intracellular domain (e.g., the co-stimulatory signaling region)) of the presently disclosed CAR (e.g., amino acids 114 to 220 of SEQ ID NO: 26 or amino acids 153 to 179 of SEQ ID NO: 26) comprises at least a portion of the sequence set forth in SEQ ID NO: 27 as provided below.

(SEQ ID NO: 27)
attgaagttatgtatcctcctccttacctagacaa
tgagaagagcaatggaaccattatccatgtgaaag
ggaaacacctttgtccaagtcccctatttcccgg
accttctaagcccttttgggtgctggtggtggttg
gtggagtcctggcttgctatagcttgctagtaaca
gtggcctttattattttctgggtgaggagtaagag
gagcaggctcctgcacagtgactacatgaacatga
ctccccgccgccccgggcccacccgcaagcattac
cagccctatgccccaccacgcgacttcgcagccta
tcgctcc

In certain embodiments, the transmembrane domain comprises a CD8 polypeptide. The CD8 polypeptide can have an amino acid sequence that is at least about 85%, about 90%, about 95%, about 96%, about 97%, about 98%, about 99% or about 100%) homologous to SEQ ID NO: 28 (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 28 which is at least 20, or at least 30, or at least 40, or at least 50, and up to 235 amino acids in length. Additionally or alternatively, in various embodiments, the CD8 polypeptide has an amino acid sequence of amino acids 1 to 235, 1 to 50, 50 to 100, 100 to 150, 150 to 200, or 200 to 235 of SEQ ID NO: 28.

(SEQ ID NO: 28)
MALPVTALLLPLALLLHAARPSQFRVSPLDRTWNL
GETVELKCQVLLSNPTSGCSWLFQPRGAAASPTF
LLYLSQNKPKAAEGLDTQRFSGKRLGDTFVLTLSD
FRRENEGYYFCSALSNSIMYFSHFVPVFLPAKPTT
TPAPRPPTPAPTIASQPLSLRPEACRPAAGGAVHT
RGLDFACDIYIWAPLAGTCGVLLLSLVITLYCNHR
NRRRVCKCPRPWKSGDKPSLSARYV

In accordance with the presently disclosed subject matter, a “CD8 nucleic acid molecule” refers to a polynucleotide encoding a CD8 polypeptide.

In certain non-limiting embodiments, a CAR can also comprise a spacer region that links the extracellular antigen-binding domain to the transmembrane domain. The spacer region can be flexible enough to allow the antigen-binding domain to orient in different directions to facilitate antigen recognition while preserving the activating activity of the CAR. In certain non-limiting embodiments, the spacer region can be the hinge region from IgG1, the CH₂CH₃ region of immunoglobulin and portions of CD3, a portion of a CD28 polypeptide (e.g., SEQ ID NO: 26), a portion of a CD8 polypeptide (e.g., SEQ ID NO: 28), a variation of any of the foregoing which is at least about 80%, at least about 85%, at least about 90%, or at least about 95% homologous thereto, or a synthetic spacer sequence. In certain non-limiting embodiments, the spacer region may have a length between about 1-50 (e.g., 5-25, 10-30, or 30-50) amino acids.

Intracellular Domain of a CAR. In certain non-limiting embodiments, an intracellular domain of the CAR can comprise a CD3ζ polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The CD3ζ polypeptide can have an amino acid sequence that is at least about 85%, about 90%, about 95%, about 96%, about 97%, about 98%, about 99% or about 100% homologous to the sequence having a NCBI Reference No: NP_932170 (SEQ ID NO: 29), or fragments thereof, and/or may optionally comprise up to one or up to two or up to three conservative amino acid substitutions.

In certain embodiments, the CD3ζ polypeptide can have an amino acid sequence that is a consecutive portion of SEQ ID NO: 30 which is at least 20, or at least 30, or at least 40, or at least 50, and up to 164 amino acids in length. Additionally or alternatively, in various embodiments, the CD3ζ polypeptide has an amino acid sequence of amino acids 1 to 164, 1 to 50, 50 to 100, 100 to 150, or 150 to 164 of SEQ ID NO: 30. In certain embodiments, the CD3ζ polypeptide has an amino acid sequence of amino acids 52 to 164 of SEQ ID NO: 30.

SEQ ID NO: 30 is provided below:

(SEQ ID NO: 30)
MKWKALFTAAILQAQLPITEAQSFGLLDPKLCYLL
DGILFIYGVILTALFLRVKFSRSADAPAYQQGQNQ
LYNELNLGRREEYDVLDKRRGRDPEMGGKPQRRKN
PQEGLYNELQKDKMAEAYSEIGMKGERRRGKGHDG
LYQGLSTATKDTYDALHMQALPPR

In certain embodiments, the CD3ζ polypeptide has the amino acid sequence set forth in SEQ ID NO: 31, which is provided below:

(SEQ ID NO: 31)
RVKFSRSAEPPAYQQGQNQLYNELNLGRREEYDVL
DKRRGRDPEMGGKPRRKNPQEGLYNELQKDKMAEA
YSEIGMKGERRRGKGHDGLYQGLSTATKDTYDALH
MQALPPR

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide (SEQ ID NO: 31) comprised in the intracellular domain of the presently disclosed CAR comprises a nucleotide sequence as set forth in SEQ ID NO: 32 as provided below.

(SEQ ID NO: 32)
AGAGTGAAGTTCAGCAGGAGCGCAGAGCCCCCCGC
GTACCAGCAGGGCCAGAACCAGCTCTATAACGAGC
TCAATCTAGGACGAAGAGAGGAGTACGATGTTTTG
GACAAGAGACGTGGCCGGGACCCTGAGATGGGGGG
AAAGCCGAGAAGGAAGAACCCTCAGGAAGGCCTGT
ACAATGAACTGCAGAAAGATAAGATGGCGGAGGCC
TACAGTGAGATTGGGATGAAAGGCGAGCGCCGGAG
GGGCAAGGGGCACGATGGCCTTTACCAGGGTCTCA
GTACAGCCACCAAGGACACCTACGACGCCCTTCAC
ATGCAGGCCCTGCCCCCTCGCG

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region. In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD 137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a UniProtKB Reference No: P41273 or NCBI Reference No: NP_001552 (SEQ ID NO: 33) or fragments thereof, and/or may optionally comprise up to one or up to two or up to three conservative amino acid substitutions.

SEQ ID NO: 33 is provided below:

(SEQ ID NO: 33)
MGNSCYNIVATLLLVLNFERTRSLQDPCSNCPAGT
FCDNNRNQICSPCPPNSFSSAGGQRTCDICRQCKG
VFRTRKECSSTSNAECDCTPGFHCLGAGCSMCEQD
CKQGQELTKKGCKDCCFGTFNDQKRGICRPWTNCS
LDGKSVLGTKERDWCGPSPADLSPGASSVTPPAPA
REPGHSPQIISFFLALTSTALLFLLFFLTLRFSWK
RGRKKLLYIFKQPFMRPVQTTQEEDGCSCRFPEEE
EGGCEL

In accordance with the presently disclosed subject matter, a “4-1BB nucleic acid molecule” refers to a polynucleotide encoding a 4-1BB polypeptide.

An OX40 polypeptide can have an amino acid sequence that is at least about 85%, about 90%, about 95%, about 96%, about 97%, about 98%, about 99% or 100% homologous to the sequence having a UniProtKB Reference No: P43489 or NCBI Reference No: NP_003318 (SEQ ID NO: 34), or fragments thereof, and/or may optionally comprise up to one or up to two or up to three conservative amino acid substitutions.

SEQ ID NO: 34 is provided below:

(SEQ ID NO: 34)
MCVGARRLGRGPCAALLLLGLGLSTVTGLHCVGDT
YPSNDRCCHECRPGNGMVSRCSRSQNTVCRPCGPG
FYNDWSSKPCKPCTWCNLRSGSERKQLCTATQDTV
CRCRAGTQPLDSYKPGVDCAPCPPGHFSPGDNQAC
KPWTNCTLAGKHTLQPASNSSDAICEDRDPPATQP
QETQGPPARPITVQPTEAWPRTSQGPSTRPVEVPG
GRAVAAILGLGLVLGLLGPLAILLALYLLRRDQRL
PPDAHKPPGGGSFRTPIQEEQADAHSTLAKI

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 35) or fragments thereof, and/or may optionally comprise up to one or up to two or up to three conservative amino acid substitutions.

SEQ ID NO: 35 is provided below:

(SEQ ID NO: 35)
MKSGLWYFFLFCLRIKVLTGEINGSANYEMFIFHN
GGVQILCKYPDIVQQFKMQLLKGGQILCDLTKTKG
SGNTVSIKSLKFCHSQLSNNSVSFFLYNLDHSHAN
YYFCNLSIFDPPPFKVTLTGGYLHIYESQLCCQLK
FWLPIGCAAFVWCILGCILICWLTKKKYSSSVHDP
NGEYMFMRATAKKSRLTDVTL

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 36) (homology herein may be determined using standard software such as BLAST or FASTA) or fragments thereof, and/or may optionally comprise up to one or up to two or up to three conservative amino acid substitutions.

SEQ ID NO: 36 is provided below:

(SEQ ID NO: 36)
MACLGFQRHKAQLNLATRTWPCTLLFFLLFIPVFC
KAMHVAQPAWLASSRGIASFVCEYASPGKATEVRV
TVLRQADSQVTEVCAATYMMGNELTFLDDSICTGT
SSGNQLTIQGLRAMDTGLYICKVELMYPPPYYLGI
GNGTQIYVIDPEPCPDSDFLLWILAAVSSGLFFYS
FLLTAVSLSKMLKKRSPLTTGVYVKMPPTEPECEK
QFQPYFIPIN

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system. In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 37) or fragments thereof, and/or may optionally comprise up to one or up to two or up to three conservative amino acid substitutions.

SEQ ID NO: 37 is provided below:

(SEQ ID NO: 37)
MQIPQAPWPVVWAVLQLGWRPGWFLDSPDRPWNPP
TFSPALLWTEGDNATFTCSFSNTSESFVLNWYRMS
PSNQTDKLAAFPEDRSQPGQDCRFRVTQLPNGRDF
HMSVVRARRNDSGTYLCGAISLAPKAQIKESLRAE
LRVTERRAEVPTAHPSPSPRPAGQFQTLVVGWGGL
LGSLVLLVWVLAVICSRAARGTIGARRTGQPLKED
PSAVPVFSVDYGELDFQWREKTPEPPVPCVPEQTE
YATIVFPSGMGTSSPARRGSADGPRSAQPLRPEDG
HCSWPL

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 38) or fragments thereof, and/or may optionally comprise up to one or up to two or up to three conservative amino acid substitutions.

SEQ ID NO: 38 is provided below:

(SEQ ID NO: 38)
MWEAQFLGLLFLQPLWVAPVKPLQPGAEVPWWAQE
GAPAQLPCSPTIPLQDLSLLRRAGVTWQHQPDSG
PPAAAPGHPLAPGPHPAAPSSW
GPRPRRYTVLSVGPGGLRSGRLPLQPRVQLDERGR
QRGDFSLWLRPARRADAGEYRAAVHLRDRALSCRL
RLRLGQASMTASPPGSLRASDWVILNCSFSRPDRP
ASVHWFRNRGQGRVPVRESPHHHLAESFLFLPQVS
PMDSGPWGCILTYRDGFNVSIMYNLTVLGLEPPTP
LTVYAGAGSRVGLPCRLPAGVGTRSFLTAKWTPPG
GGPDLLVTGDNGDFTLRLEDVSQAQAGTYTCHIHL
QEQQLNATVTLAIITVTPKSFGSPGSLGKLLCEVT
PVSGQERFVWSSLDTPSQRSFSGPWLEAQEAQLLS
QPWQCQLYQGERLLGAAVYFTELSSPGAQRSGRAP
GALPAGHLLLFLILGVLSLLLLVTGAFGFHLWRRQ
WRPRRFSALEQGIHPPQAQSKIEELEQEPEPEPEP
EPEPEPEPEPEQL

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref No.: Q9BZW8.2 (SEQ ID NO: 39) or fragments thereof, and/or may optionally comprise up to one or up to two or up to three conservative amino acid substitutions.

SEQ ID NO: 39 is provided below:

(SEQ ID NO: 39)
MLGQWTLILLLLLKVYQGKGCQGSADHWSISGVPL
QLQPNSIQTKVDSIAWKKLLPSQNGFHHILKWENG
SLPSNTSNDRFSFIVKNLSLLIKAAQQQDSGLYCL
EVTSISGKVQTATFQVFVFESLLPDKVEKPRLQGQ
GKILDRGRCQVALSCLVSRDGNVSYAWYRGSKLIQ
TAGNLTYLDEEVDINGTHTYTCNVSNPVSWESHTL
NLTQDCQNAHQEFRFWPFLVIIVILSALFLGTLAC
FCVWRRKRKEKQSETSPKEFLTIYEDVKDLKTRRN
HEQEQTFPGGGSTIYSMIQSQSSAPTSQEPAYTLY
SLIQPSRKSGSRKRNHSPSFNSTIYEVIGKSQPKA
QNPARLSRKELENFDVYS

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8+ cancer-specific T cells. BTLA has also been designated as CD272 (cluster of differentiation 272).

In accordance with the presently disclosed subject matter, a BTLA polypeptide can have an amino acid sequence that is at least about 85%>, about 90%, about 95%, about 96%, about 97%, about 98%, about 99% or about 100% homologous to UniProtKB/Swiss-Prot Ref. No.: Q7Z6A9.3 (SEQ ID NO: 40) or fragments thereof, and/or may optionally comprise up to one or up to two or up to three conservative amino acid substitutions.

SEQ ID NO: 40 is provided below:

(SEQ ID NO: 40)
MKTLPAMLGTGKLFWVFFLIPYLDIWNIHGKESCD
VQLYIKRQSEHSILAGDPFELECPVKYCANRPHVT
WCKLNGTTCVKLEDRQTSWKEEKNISFFILHFEPV
LPNDNGSYRCSANFQSNLIESHSTTLYVTDVKSAS
ERPSKDEMASRPWLLYRLLPLGGLPLLITTCFCLF
CCLRRHQGKQNELSDTAGREINLVDAHLKSEQTEA
STRQNSQVLLSETGIYDNDPDLCFRMQEGSEVYSN
PCLEENKPGIVYASLNHSVIGPNSRLARNVKEAPT
EYASICVRS

In accordance with the presently disclosed subject matter, a “BTLA nucleic acid molecule” refers to a polynucleotide encoding a BTLA polypeptide.

Exemplary CAR and prodrug converting enzyme constructs. In certain embodiments, the CAR and prodrug converting enzyme are expressed as single polypeptide linked by a self-cleaving linker, such as a P2A linker. In certain embodiments, the CAR and prodrug converting enzyme are expressed as two separate polypeptides.

In certain embodiments, the CAR comprises an extracellular antigen-binding region that comprises a human scFv that specifically binds to a human tumor antigen, a transmembrane domain comprising a CD28 polypeptide, and an intracellular domain comprising a CD3ζ polypeptide and a co-stimulatory signaling region that comprises a 4-1BB polypeptide. The CAR may comprise a signal peptide or a leader covalently joined to the N-terminus of the extracellular antigen-binding domain. The signal peptide may comprise amino acids having the sequence set forth in SEQ ID NO: 22 or SEQ ID NO: 24. In certain embodiments, the human scFv is selected from the group consisting of an anti-CD19 scFv, and anti-WT1 scFv, and anti-PRAME scFv.

In some embodiments, the nucleic acid encoding the CAR and the prodrug converting enzyme (e.g., CPG2 or β-lactamase) is operably linked an inducible promoter. In some embodiments, the nucleic acid encoding the CAR and the prodrug converting enzyme (e.g., CPG2 or β-lactamase) is operably linked a constitutive promoter. In some embodiments, the nucleic acid encoding the CAR and the nucleic acid encoding prodrug converting enzyme (e.g., CPG2 or β-lactamase) are operably linked to two separate promoters. In some embodiments, the nucleic acid encoding the CAR is operably linked a constitutive promoter and the prodrug converting enzyme (e.g., CPG2 or β-lactamase) is operably linked a constitutive promoter. In some embodiments, the nucleic acid encoding the CAR is operably linked a constitutive promoter and the prodrug converting enzyme (e.g., CPG2 or β-lactamase) is operably linked an inducible promoter.

In some embodiments, the inducible promoter is a synthetic Notch promoter that is activatable in a CAR T cell, where the intracellular domain of the CAR contains a transcriptional regulator that is released from the membrane when engagement of the CAR with the tumor antigen induces intramembrane proteolysis (see, e.g. Morsut et al., Cell 164(4): 780-791 (2016). Accordingly, transcription of the prodrug converting enzyme is induced upon binding of the engineered immune cell with the tumor antigen.

The presently disclosed subject matter also provides isolated nucleic acid molecules encoding the CAR/prodrug converting enzyme constructs described herein or a functional portion thereof. In certain embodiments, the isolated nucleic acid molecule encodes an anti-CD19-targeted CAR comprising a human scFv that specifically binds to a human CD19 polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and a CPG2 or β-lactamase polypeptide fused to a signal peptide and a transmembrane domain comprising a CD8 polypeptide.

In certain embodiments, the isolated nucleic acid molecule encodes an anti-CD19-targeted CAR comprising a human scFv that specifically binds to a human CD19 polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and a CPG2 or β-lactamase polypeptide fused to a signal peptide.

In certain embodiments, the isolated nucleic acid molecule encodes an anti-CD19-targeted CAR comprising a human scFv that specifically binds to a human CD19 polypeptide fused to a synthetic Notch transmembrane domain and an intracellular cleavable transcription factor. In certain embodiments, the isolated nucleic acid molecule encodes a CPG2 or 0-lactamase enzyme inducible by release of the transcription factor of a synthetic Notch system.

In certain embodiments, the isolated nucleic acid molecule encodes a functional portion of a presently disclosed CAR constructs. As used herein, the term “functional portion” refers to any portion, part or fragment of a CAR, which portion, part or fragment retains the biological activity of the targeted CAR (the parent CAR). For example, functional portions encompass the portions, parts or fragments of a tumor antigen-targeted CAR that retains the ability to recognize a target cell, to treat a disease, e.g., solid tumor, to a similar, same, or even a higher extent as the parent CAR. In certain embodiments, an isolated nucleic acid molecule encoding a functional portion of a tumor antigen-targeted CAR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CAR.

### Engineered Immune Cells of the Present Technology

The present disclosure provides engineered immune cells expressing a prodrug converting enzyme and a T-cell receptor (e.g., a CAR) or other ligand that comprises an extracellular antigen-binding domain, a transmembrane domain and an intracellular domain, where the extracellular antigen-binding domain specifically binds tumor antigen, including a tumor receptor or ligand, as described above. In certain embodiments, immune cells can be transduced with a presently disclosed CAR/prodrug converting enzyme constructs such that the cells express the CAR and the prodrug converting enzyme. The presently disclosed subject matter also provides methods of using such cells for the treatment of a tumor. The engineered immune cells of the presently disclosed subject matter can be cells of the lymphoid lineage or myeloid lineage. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., T_{EM} cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the CAR-expressing T cells express Foxp3 to achieve and maintain a T regulatory phenotype.

Natural killer (NK) cells can be lymphocytes that are part of cell-mediated immunity and act during the innate immune response. NK cells do not require prior activation in order to perform their cytotoxic effect on target cells.

The engineered immune cells of the presently disclosed subject matter can express an extracellular antigen-binding domain (e.g., a human scFv, a Fab that is optionally crosslinked, or a F(ab)₂) that specifically binds to a tumor antigen, for the treatment of cancer, e.g., for treatment of solid tumor. Such engineered immune cells can be administered to a subject (e.g., a human subject) in need thereof for the treatment of cancer. In some embodiments, the immune cell is a lymphocyte, such as a T cell, a B cell or a natural killer (NK) cell. In certain embodiments, the engineered immune cell is a T cell. The T cell can be a CD4⁺ T cell or a CD8⁺ T cell. In certain embodiments, the T cell is a CD4⁺ T cell. In certain embodiments, the T cell is a CD8⁺ T cell.

A presently disclosed engineered immune cells can further include at least one recombinant or exogenous co-stimulatory ligand. For example, a presently disclosed engineered immune cells can be further transduced with at least one co-stimulatory ligand, such that the engineered immune cells co-expresses or is induced to co-express the tumor antigen-targeted CAR and the at least one co-stimulatory ligand. The interaction between the tumor antigen-targeted CAR and at least one co-stimulatory ligand provides a non-antigen-specific signal important for full activation of an immune cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta O-Tβ), CD257/B cell-activating factor (B AFF)/Bly s/THANK/Tall-1, glucocorticoid-induced TNF Receptor ligand (GITRL), and T F-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the engineered immune cell comprises one recombinant co-stimulatory ligand that is 4-1BBL. In certain embodiments, the engineered immune cell comprises two recombinant co-stimulatory ligands that are 4-1BBL and CD80. CARs comprising at least one co-stimulatory ligand are described in U.S. Pat. No. 8,389,282, which is incorporated by reference in its entirety.

Furthermore, a presently disclosed engineered immune cells can further comprise at least one exogenous cytokine. For example, a presently disclosed engineered immune cell can be further transduced with at least one cytokine, such that the engineered immune cells secretes the at least one cytokine as well as expresses the tumor antigen-targeted CAR. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The engineered immune cells can be generated from peripheral donor lymphocytes, e.g., those disclosed in Sadelain, M., et al., Nat Rev Cancer 3:35-45 (2003) (disclosing peripheral donor lymphocytes genetically modified to express CARs), in Morgan, R. A. et al., Science 314: 126-129 (2006) (disclosing peripheral donor lymphocytes genetically modified to express a full-length tumor antigen-recognizing T cell receptor complex comprising the α and β heterodimer), in Panelli et al. J Immunol 164:495-504 (2000); Panelli et al. J Immunol 164:4382-4392 (2000) (disclosing lymphocyte cultures derived from tumor infiltrating lymphocytes (TILs) in tumor biopsies), and in Dupont et al. Cancer Res 65:5417-5427 (2005); Papanicolaou et al. Blood 102:2498-2505 (2003) (disclosing selectively in v/Yro-expanded antigen-specific peripheral blood leukocytes employing artificial antigen-presenting cells (AAPCs) or pulsed dendritic cells). The engineered immune cells (e.g., T cells) can be autologous, non-autologous (e.g., allogeneic), or derived in vitro from engineered progenitor or stem cells.

In certain embodiments, a presently disclosed engineered immune cells (e.g., T cells) expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a presently disclosed tumor antigen-targeted CAR and/or prodrug converting enzyme.

For example, the higher the CAR expression level in an engineered immune cell, the greater cytotoxicity and cytokine production the engineered immune cell exhibits. An engineered immune cell (e.g., T cell) having a high tumor antigen-targeted CAR expression level can induce antigen-specific cytokine production or secretion and/or exhibit cytotoxicity to a tissue or a cell having a low expression level of tumor antigen-targeted CAR, e.g., about 2,000 or less, about 1,000 or less, about 900 or less, about 800 or less, about 700 or less, about 600 or less, about 500 or less, about 400 or less, about 300 or less, about 200 or less, about 100 or less of tumor antigen binding sites/cell. Additionally or alternatively, the cytotoxicity and cytokine production of a presently disclosed engineered immune cell (e.g., T cell) are proportional to the expression level of tumor antigen in a target tissue or a target cell. For example, the higher the expression level of human tumor antigen in the target, the greater cytotoxicity and cytokine production the engineered immune cell exhibits.

As described herein, the co-expression of prodrug converting enzyme increases the cytotoxic effect in the CAR T cells by converting a prodrug compound into an active drug at the target site. In certain embodiments, an engineered immune cell of the present disclosure exhibits a cytotoxic effect against tumor antigen-expressing cells that is at least about 2-times, about 3-times, about 4-times, about 5-times, about 6-times, about 7-times, about 8-times, about 9-times, about 10-times, about 20-times, about 30-times, about 40-times, about 50-times, about 60-times, about 70-times, about 80-times, about 90-times, or about 100-times, the cytotoxic effect in the absence of the prodrug converting enzyme.

The unpurified source of immune cells may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Preferably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Preferably, the cells are collected in a medium comprising 2% fetal calf serum (FCS) or 0.2% bovine serum albumin (BSA) or any other suitable, preferably sterile, isotonic medium.

In some embodiments, the engineered immune cells comprise one or more additional modifications. For example, in some embodiments, the engineered immune cells comprise and express (is transduced to express) an antigen recognizing receptor that binds to a second antigen that is different than selected tumor antigen. The inclusion of an antigen recognizing receptor in addition to a presently disclosed CAR on the engineered immune cell can increase the avidity of the CAR or the engineered immune cell comprising thereof on a targeted cell, especially, the CAR is one that has a low binding affinity to a particular tumor antigen, e.g., a K_{d} of about 2×10⁻⁸ M or more, about 5×10⁻⁸ M or more, about 8×10⁻⁸ M or more, about 9×10⁻⁸ M or more, about 1×10⁻⁷ M or more, about 2×10⁻⁷ M or more, or about 5×10⁻⁷ M or more.

In certain embodiments, the antigen recognizing receptor is a chimeric co-stimulatory receptor (CCR). CCR is described in Krause, et al., J. Exp. Med. 188(4):619-626(1998), and US20020018783, the contents of which are incorporated by reference in their entireties. CCRs mimic co-stimulatory signals, but unlike, CARs, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and costimulation to generate T cells that eliminate target cells that express a combination of antigens while sparing cells that express each antigen individually (Kloss et al., Nature Biotechnology 31(1):71-75 (2013)). With this approach, T-cell activation requires CAR-mediated recognition of one antigen, whereas costimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen. In certain embodiments, the CCR comprises an extracellular antigen-binding domain that binds to an antigen different than selected tumor antigen, a transmembrane domain, and a co-stimulatory signaling region that comprises at least one co-stimulatory molecule, including, but not limited to, CD28, 4-1BB, OX40, ICOS, PD-1, CTLA-4, LAG-3, 2B4, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-IBB. In certain embodiments, the co-stimulatory signaling region of the CCR comprises two co-stimulatory signaling molecules. In certain embodiments, the two co-stimulatory signaling molecules are CD28 and 4-IBB. A second antigen is selected so that expression of both selected tumor antigen and the second antigen is restricted to the targeted cells (e.g., cancerous tissue or cancerous cells). Similar to a CAR, the extracellular antigen-binding domain can be a scFv, a Fab, a F(ab)₂; or a fusion protein with a heterologous sequence to form the extracellular antigen-binding domain. In certain embodiments, the CCR comprises a scFv that binds to CD 138, transmembrane domain comprising a CD28 polypeptide, and a co-stimulatory signaling region comprising two co-stimulatory signaling molecules that are CD28 and 4-IBB.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells, e.g., myeloma cells. The truncated CAR functions as an adhesion molecule that enhances the avidity of a presently disclosed CAR, especially, one that has a low binding affinity to tumor antigen, thereby improving the efficacy of the presently disclosed CAR or engineered immune cell (e.g., T cell) comprising thereof. In certain embodiments, the truncated CAR comprises an extracellular antigen-binding domain that binds to CD 138, a transmembrane domain comprising a CD8 polypeptide. A presently disclosed T cell comprises or is transduced to express a presently disclosed CAR targeting tumor antigen and a truncated CAR targeting CD138. In certain embodiments, the targeted cells are solid tumor cells.

In some embodiments, the engineered immune cells are further modified to suppress expression of one or more genes. In some embodiments, the engineered immune cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered immune cells are modified to disrupt or reduce expression of an endogenous T-cell receptor gene (see, e.g. WO 2014153470, which is incorporated by reference in its entirety). In some embodiments, the engineered immune cells are modified to result in disruption or inhibition of PD1, PDL-1 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### Vectors

Many expression vectors are available and known to those of skill in the art and can be used for expression of polypeptides provided herein. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g. a hexa-his tag or a myc tag, hemagglutinin tag or a tag for purification, for example, a GST fusion, and a sequence for directing protein secretion and/or membrane association.

Expression of the antibodies or antigen-binding fragments thereof can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al., Cell 22:787-797(1980)), the herpes thymidine kinase promoter (Wagner et al., Proc. Natl. Acad. Sci. USA 75: 1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510: 1 15-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515: 115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7: 1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254: 1372-1378 (1986)).

In addition to the promoter, the expression vector typically contains a transcription unit or expression cassette that contains all the additional elements required for the expression of the antibody, or portion thereof, in host cells. A typical expression cassette contains a promoter operably linked to the nucleic acid sequence encoding the antibody chain and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of engineered immune cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the tumor antigen-targeted CAR and the prodrug converting enzyme can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide tumor antigen-targeted CAR and the prodrug converting enzyme expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al. Mol. Cell. Biol. 5:431-437 (1985)); PA317 (Miller, et al. Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni, et al. Blood 80: 1418-1422(1992), or culturing with viral supernatant alone or concentrated vector stocks with or without appropriate growth factors and polycations, e.g., by the method of Xu, et al. Exp. Hemat. 22:223-230 (1994); and Hughes, et al. J. Clin. Invest. 89: 1817 (1992).

Transducing viral vectors can be used to express a co-stimulatory ligand and/or secretes a cytokine (e.g., 4-1BBL and/or IL-12) in an engineered immune cell. Preferably, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61(1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing a presently disclosed tumor antigen-targeted CAR is a retroviral vector, e.g., an oncoretroviral vector.

Non-viral approaches can also be employed for the expression of a protein in cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J Med. Sci. 298:278, (1989); Staubinger et al., Methods in Enzymology 101:512 (1983)), asialoorosomucoid-polylysine conjugation (Wu et al., Journal of Biological Chemistry 263: 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor la enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes. VI. Polypeptides and Analogs and Polynucleotides

Also included in the presently disclosed subject matter are extracellular antigen-binding domains that specifically binds to a tumor antigen (e.g., human tumor antigen) (e.g., an scFv (e.g., a human scFv), a Fab, or a (Fab)₂), CD3ζ, CD8, CD28, etc. polypeptides or fragments thereof, and polynucleotides encoding thereof that are modified in ways that enhance their anti-tumor activity when expressed in an engineered immune cell. The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%), about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the invention. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the antineoplastic activity of the original polypeptide when expressed in an engineered immune cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular antigen-binding domain that specifically binds to tumor antigen (e.g., human tumor antigen) (e.g., an scFv (e.g., a human scFv), a Fab, or a (Fab)₂), CD3, CD8, CD28) can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### Administration

Engineered immune cells expressing a tumor antigen-targeted CAR and a prodrug converting enzyme can be provided systemically or directly to a subject for treating or preventing a neoplasia. In certain embodiments, engineered immune cells are directly injected into an organ of interest (e.g., an organ affected by a neoplasia). Alternatively or additionally, the engineered immune cells are provided indirectly to the organ of interest, for example, by administration into the circulatory system (e.g., the tumor vasculature). Expansion and differentiation agents can be provided prior to, during or after administration of cells and compositions to increase production of T cells in vitro or in vivo.

Engineered immune cells of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered immune cells can comprise a purified population of cells. Those skilled in the art can readily determine the percentage of engineered immune cells in a cell population using various well-known methods, such as fluorescence activated cell sorting (FACS). The ranges of purity in cell populations comprising engineered immune cells can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The engineered immune cells can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, pharmaceutical compositions comprising engineered immune cells expressing a tumor antigen-targeted CAR and a prodrug converting enzyme with a pharmaceutically acceptable carrier are administered to a subject. Administration can be autologous or non-autologous. For example, engineered immune cells expressing a tumor antigen-targeted CAR and a prodrug converting enzyme and compositions comprising thereof can be obtained from one subject, and administered to the same subject or a different, compatible subject. Peripheral blood derived engineered T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition comprising engineered immune cells expressing a tumor antigen-targeted CAR and a prodrug converting enzyme, it can be formulated in a unit dosage injectable form (solution, suspension, emulsion).

### Formulations

Engineered immune cells expressing a tumor antigen-targeted CAR and prodrug converting enzyme and compositions comprising the same can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating compositions comprising an engineered immune cells expressing a tumor antigen-targeted CAR and prodrug converting enzyme, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the engineered immune cells disclosed herein.

The compositions can be isotonic, i.e., they can have the same osmotic pressure as blood and lacrimal fluid. The desired isotonicity of compositions comprising an engineered immune cells expressing a tumor antigen-targeted CAR and prodrug converting enzyme may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered immune cells described herein. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered immune cells is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ of the engineered immune cells disclosed herein are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more of the engineered immune cells disclosed herein are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Methods for Therapy

In a related aspect, a method for treating cancer in a subject in need thereof is provided, where the method includes administering an effective amount of an engineered immune cell (e.g., any embodiment of the SEAKER disclosures) and administering an effective amount of a prodrug compound of any embodiment disclosed herein, wherein the engineered immune cell includes a prodrug converting enzyme and/or a nucleic acid encoding the prodrug converting enzyme and a receptor that binds to a target antigen and/or nucleic acid encoding the receptor.

The amount of the engineered immune cells (any aspect or embodiment as disclosed in the SEAKER disclosures) administered is an amount effective in producing the desired effect, for example, treatment of a cancer or one or more symptoms of a cancer. An effective amount can be provided in one or a series of administrations of the engineered immune cells provided herein. An effective amount can be provided in a bolus or by continuous perfusion. For adoptive immunotherapy using antigen-specific T cells, cell doses in the range of about 10⁶ to about 10¹⁰ are typically infused. Co-expression of the prodrug converting enzyme as disclosed herein, may permit lower doses of the engineered immune cells to be administered, e.g., about 10⁴ to about 10⁸. Upon administration of the engineered immune cells into the subject, the engineered immune cells are induced that are specifically directed against one tumor antigen. “Induction” of T cells can include inactivation of antigen-specific T cells such as by deletion or anergy. Inactivation is particularly useful to establish or reestablish tolerance such as in autoimmune disorders. The engineered immune cells can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the engineered immune cells and the compositions comprising thereof are intravenously administered to the subject in need. Methods for administering cells for adoptive cell therapies, including, for example, donor lymphocyte infusion and CAR T cell therapies, and regimens for administration are known in the art and can be employed for administration of the engineered immune cells provided herein.

The presently disclosed subject matter provides various methods of using engineered immune cells (e.g., T cells) expressing a tumor antigen-targeted receptor (e.g., a CAR) and a prodrug converting enzyme. For example, the presently disclosed subject matter provides methods of reducing tumor burden in a subject via use of an engineered immune cell with a compound of the present technology. In one non-limiting example, the method of reducing tumor burden may include administering an effective amount of engineered immune cells (of any aspect or embodiment of the SEAKER disclosures) to the subject and administering a prodrug compound of the present technology for conversion by the expressed prodrug converting enzyme, thereby inducing tumor cell death in the subject. In some embodiments, the engineered immune cells and the prodrug are administered at different times. For example, in some embodiments, the engineered immune cells are administered and then the prodrug is administered. In some embodiments, the prodrug is administered 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 26 hours, 48 hours or longer after the administration of the engineered immune cells.

The engineered immune cells in combination with a prodrug compound of any embodiment disclosed herein may reduce the number of tumor cells, reduce tumor size, and/or eradicate the tumor in the subject. Non-limiting examples of suitable tumors include adrenal cancers, bladder cancers, blood cancers, bone cancers, brain cancers, breast cancers, epithelial carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, cancers with activated EGRF receptors, cancers with other activated receptor tyrosine kinases, and metastases of any one or more thereof. In any embodiment disclosed herein, it may be the cancer is a relapsed or refractory cancer. In any embodiment disclosed herein, it may be the cancer is resistant to one or more cancer therapies, e.g., one or more chemotherapeutic drugs.

Additionally or alternatively, in some embodiments, the methods of the present technology further comprise administering an additional cancer therapy. Examples of additional cancer therapies include, but are not limited to, chemotherapy, radiation therapy, immunotherapy, monoclonal antibodies, anti-cancer nucleic acids or proteins, anti-cancer viruses or microorganisms, and any combinations thereof.

Additionally or alternatively, in some embodiments, the methods of the present technology further comprises administering a cytokine to the subject. The cytokine may be administered prior to, during, or subsequent to administration of the one or more engineered immune cells. Examples of cytokines include, but are not limited to interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

Additionally or alternatively, in some embodiments of the methods disclosed herein, the prodrug converting enzyme is a carboxypeptidase, e.g., CPG2. In certain embodiments, the methods disclosed herein does not comprise use of recombinant enzymes. In other embodiments, the methods disclosed herein comprise the use of recombinant enzymes.

### Articles of Manufacture and Kits

The presently disclosed subject matter provides kits for the treatment or prevention of a neoplasia (e.g., solid tumor). The kit includes a compound of the present technology and instructions for use according to a method of treatment/use of any embodiment disclosed herein and/or any embodiment disclosed in the SEAKER disclosures. The kit of any embodiment may further include a therapeutic or prophylactic composition containing an effective amount of an engineered immune cell comprising a tumor antigen-targeted receptor (e.g., a CAR) and prodrug converting enzyme in unit dosage form. In any embodiment disclosed herein, it may be the cells further expresses at least one co-stimulatory ligand. In any embodiment disclosed herein, it may be the kit includes a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

The kit may further be provided together with instructions for administering a compound of any embodiment herein (and optionally an engineered immune cell of any embodiment disclosed herein and/or in the SEAKER disclosures) to a subject having or at risk of developing a neoplasia (e.g., solid tumor). Such instructions will generally include information about the use of the composition for the treatment or prevention of a neoplasia (e.g., solid tumor). In other embodiments, the instructions include at least one of the following: description of the therapeutic agent; dosage schedule and administration for treatment or prevention of a neoplasia (e.g., solid tumor) or symptoms thereof; precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The examples herein are provided to illustrate advantages of the present technology and to further assist a person of ordinary skill in the art with preparing or using the compounds of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described herein. The variations, aspects, or embodiments described above may also further each include or incorporate the variations of any or all other variations, aspects, or embodiments of the present technology.

### Example 1. Exemplary Synthesis of a Prodrug of the Present Technology

This example describes the synthesis of an exemplary prodrug based on gefitinib for cleavage by the bacterial hydrolase enzyme CPG2. The synthesis of this prodrug (APdMG-Glu) is summarized in Scheme 1 below (where APdMG-Glu is also referred to as S4) and further detailed thereafter.

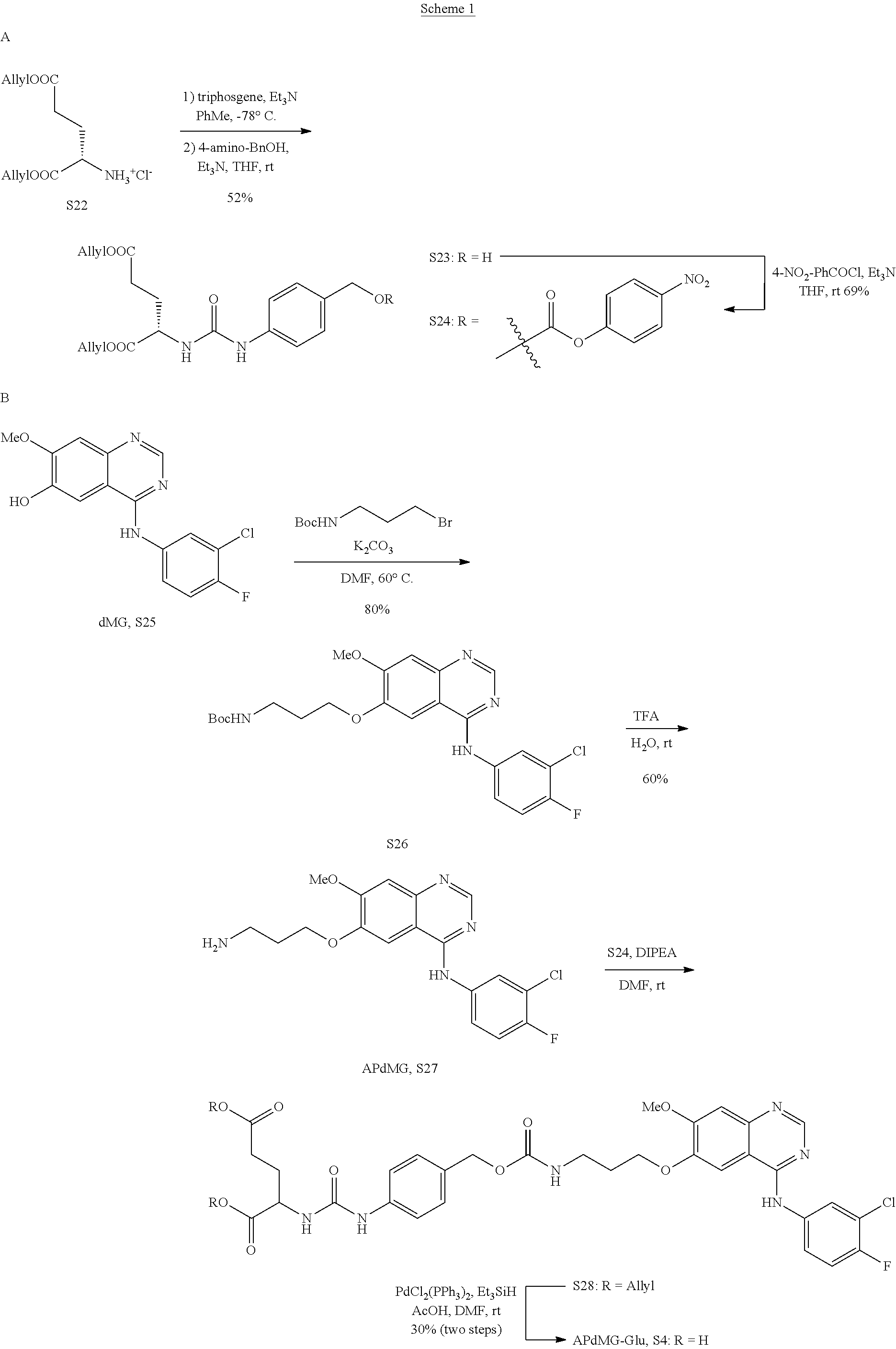

### Diallyl ((4-(Hydroxymethyl)phenyl)carbamoyl)-L-glutamate (S23)

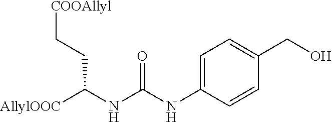

In a 50-mL roundbottom flask, L-glutamic acid dimethyl ester hydrochloride S22 (1000 mg, 2.50 mmol, 1.0 equiv) was dissolved in 25 mL toluene and cooled to −78° C. Triethylamine (732 μL, 5.26 mmol, 2.1 equiv) and triphosgene (371 mg, 1.25 μmol, 0.5 equiv) were added and the mixture was stirred at −78° C. for 30 min. The reaction mixture was filtered and concentrated by rotary evaporation. The crude residue was dissolved in THF (37.5 mL). 4-Aminobenzyl alcohol (339 mg, 2.75 mmol, 1.1 equiv) and triethylamine (732 μL, 5.26 mmol, 2.1 equiv) were added and the mixture was stirred at rt for 1 h. The reaction mixture was concentrated by rotary evaporation and the crude residue was partitioned between brine (35 mL) and EtOAc (35 mL). The organic extracts were washed with water (1×20 mL) and brine (1×20 mL), dried (MgSO₄), filtered, and concentrated by rotary evaporation. Purification by silica flash chromatography (3:1 hexanes/EtOAc→1:1 hexanes/EtOAc) yielded PABA-Glu(OAllyl)₂ S23 (489 mg, 52%) as a clear oil. Analytical data for S23 were in agreement with those reported in Niculescu-Duvaz, D.; et al. J. Med. Chem. 1998, 41, 5297-5309.

### Diallyl ((4-((((4-nitrophenoxy)carbonyl)oxy)methyl)phenyl)carbamoyl)-L-glutamate (S24)

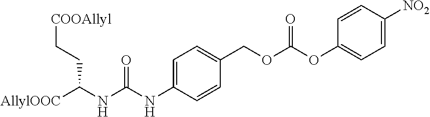

In a 50-mL roundbottom flask, PABA-Glu(OAllyl)₂ S23 (468 mg, 1.24 mmol, 1.0 equiv) was dissolved in 12.4 mL THE at rt. Triethylamine (364 μL, 2.61 mmol, 2.1 equiv) and 4-nitrophenyl chloroformate (526 mg, 2.61 mmol, 2.1 equiv) were added and the mixture was stirred at rt for 2 h. The reaction mixture was filtered and concentrated by rotary evaporation. Purification by silica flash chromatography (3:1 hexanes/EtOAc→1:1 hexanes/EtOAc) yielded PNP-activated PABA-Glu S24 (450 mg, 69%) as a white solid. Analytical data for S24 were in agreement with those reported in Niculescu-Duvaz, D.; et al. J. Med. Chem. 1998, 41, 5297-5309 and Niculescu-Duvaz, D.; et al. J. Med. Chem. 2003, 46, 1690-1705.

### tert-Butyl (3-((4-((3-chloro-4-fluorophenyl)amino)-7-methoxyquinazolin-6-yl)oxy)propyl)carbamate (S26)

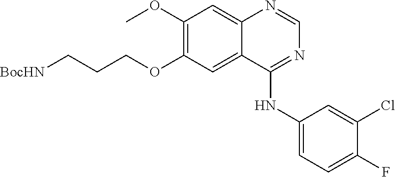 [SMILES: COc1cc2ncnc(Nc3ccc(F)c(Cl)c3)c2cc1OCCCNC(=O)OC(C)(C)C]

In a 50-mL roundbottom flask, dMG S25 (Toronto Research Chemicals, 500 mg, 1.56 mmol, 1.0 equiv) was dissolved in 15.6 mL DMF and heated to 60° C. Potassium carbonate (323 mg, 2.34 mmol, 1.5 equiv) and 3-(boc-amino)propyl bromide (409 mg, 1.72 mmol, 1.1 equiv) were added and the mixture was stirred at 60° C. for 16 h, until complete conversion had occurred as judged by LC-MS. The reaction mixture was concentrated by rotary evaporation leaving a brown oil. Purification by silica flash chromatography (100% CH₂Cl₂→5% MeOH in CH₂Cl₂) yielded N-Boc-dMG S26 (596 mg, 80%) as a white solid. Analytical data for S26 were in agreement with those reported in Yoo, B.; et al. Bioorg. Med. Chem. 2015, 23, 7119-7130.

### 6-(3-Aminopropoxy)-N-(3-chloro-4-fluorophenyl)-7-methoxyquinazolin-4-amine (APdMG, S27)

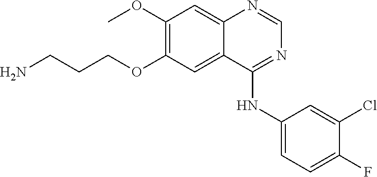 [SMILES: COc1cc2ncnc(Nc3ccc(F)c(Cl)c3)c2cc1OCCCN]

In a 25-mL roundbottom flask, N-Boc-dMG S26 (596 mg, 1.25 mmol, 1.0 equiv) was dissolved in 5.9 mL 9:1 TFA:water at rt for 30 min. The solution was cooled to 0° C., then MeOH (5 mL) was added and the mixture was concentrated by rotary evaporation. The residue was evaporated from MeOH (5 mL) two more times to yield a light yellow oil. The oil was washed with diethyl ether, dissolved in a solution of H₂O/CH₃CN, frozen, and lyophilized to yield APdMG S27 (283 mg, 60%) as a tan solid that was used without further purification. Analytical data for S27 were in agreement with those reported in Yoo, B.; et al. Bioorg. Med. Chem. 2015, 23, 7119-7130.

### Diallyl ((4-((((3-((4-((3-chloro-4-fluorophenyl)amino)-7-methoxyquinazolin-6-yl)oxy)propyl)carbamoyl)oxy)methyl)phenyl)carbamoyl)-L-glutamate (S28)

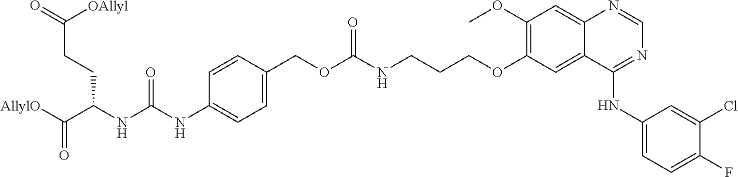

In a 25-mL roundbottom flask, PNP-activated PABA-Glu S24 (313 mg, 831 μmol, 1.0 equiv) was dissolved 8.3 mL DMF at rt. ApDMG S27 (450 mg, 831 μmol, 1.0 equiv) and DIPEA (723 μL, 4.15 mmol, 5 equiv) were added and the mixture was stirred at rt for 4 h. The mixture was poured into satd aq NH₄Cl (20 mL) and extracted with EtOAc (2×20 mL). The combined organic extracts were washed satd aq NH₄Cl (1×20 mL), brine (1×20 mL), and water (1×20 mL). The combined organic extracts were dried (MgSO₄), filtered, and concentrated by rotary evaporation to afford the crude product as a yellow oil. Purification by silica flash chromatography (2.5% MeOH in CH₂Cl₂→5% MeOH in CH₂Cl₂) yielded APdMG-Glu(OAllyl)₂ S28 (388 mg, 60%) as a light yellow oil. TLC: R_{f} 0.15 (5% MeOH in CH₂Cl₂). IR (ZnSe, ATR): 3377 (N—H st), 2506, 2229, 2073, 1786 (C═O st), 1679 (C═O st), 1431, 1338 (SO2 asym st), 1201, 1123 (SO2 sym st), 979. ¹H NMR (500 MHz, Methanol-d₄) δ 8.72 (s, 1H), 7.94 (dd, J=6.6, 2.6 Hz, 1H), 7.90 (s, 1H), 7.70-7.62 (m, 1H), 7.35 (t, J=8.9 Hz, 1H), 7.28 (d, J=8.2 Hz, 2H), 7.21 (d, J=8.7 Hz, 3H), 6.05-5.86 (m, 2H), 5.44-5.15 (m, 4H), 4.99 (s, 2H), 4.66 (tt, J=3.9, 1.6 Hz, 2H), 4.58 (dt, J=5.8, 1.4 Hz, 2H), 4.43 (dd, J=8.6, 5.3 Hz, 1H), 4.26 (t, J=6.0 Hz, 2H), 4.03 (s, 3H), 3.40 (t, J=6.4 Hz, 2H), 2.50 (td, J=7.3, 3.9 Hz, 2H), 2.29-2.15 (m, 1H), 2.10 (q, J=6.3 Hz, 2H), 2.01 (ddd, J=13.2, 8.5, 6.8 Hz, 1H). ¹³C NMR (126 MHz, Methanol-d₄) δ 173.9, 173.6, 160.0, 159.2, 159.0, 158.5, 157.4, 156.5, 152.1, 149.6, 140.6, 136.9, 135.1, 135.1, 133.6, 133.3, 132.3, 129.7, 127.8, 125.9, 125.8, 121.8, 121.7, 119.8, 118.8, 118.4, 117.9, 117.7, 108.8, 104.5, 100.5, 68.8, 67.2, 66.9, 66.3, 57.4, 53.5, 49.5, 49.3, 49.3, 49.2, 49.2, 49.1, 49.0, 48.9, 48.8, 48.7, 48.6, 48.5, 39.1, 31.1, 30.3, 28.4. HRMS (ESI) m z calcd for C₃₈H₄₁ClFN₆O₉ ([M+H]⁺) 779.2608; found (rel int) 779.2604 ([M+H]⁺, 100).

### ((4-((((3-((4-((3-chloro-4-fluorophenyl)amino)-7-methoxyquinazolin-6-yl)oxy)propyl) carbamoyl)oxy)methyl)phenyl)carbamoyl)-L-glutamic acid (APdMG-Glu, S4)

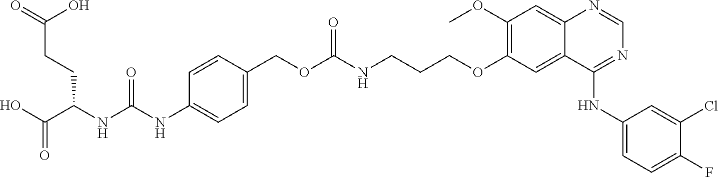 [SMILES: COc1cc2ncnc(Nc3ccc(F)c(Cl)c3)c2cc1OCCCNC(=O)OCc1ccc(NC(=O)N[C@@H](CCC(=O)O)C(=O)O)cc1]

In a 25 mL roundbottom flask, APdMG-Glu(OAllyl)₂ S28 (182 mg, 234 mol, 1.0 equiv) was dissolved in 4.7 mL DMF at rt. Glacial acetic acid (40.0 μL, 700 μmol, 3.0 equiv), bis(triphenylphosphine)palladium(II) chloride (41.0 mg, 58.4 μmol, 0.25 equiv) and triethylsilane (112 μL, 700 μmol, 3 equiv) were added and the mixture was stirred at rt for 2 h. The mixture was poured into water (20 mL), acidity adjusted to pH 7 with trifluoroacetic acid, and extracted with EtOAc (2×20 mL). The combined organic extracts were washed with water (3×10 mL), dried (MgSO₄), filtered, and concentrated by rotary evaporation to afford the crude product as a yellow oil. Purification via preparative HPLC (C18; 10×150 mm; gradient 20-95% CH₃CN in H₂O with 0.1% TFA over 20 min; 15 mL/min) and lyophilization yielded ApDMG-Glu S4 (163 mg, 30% two steps) as a white powder. IR (ZnSe, ATR): 3377 (N—H st), 2506, 2229, 2073, 1786 (C═O st), 1679 (C═O st), 1431, 1338 (SO2 asym st), 1201, 1123 (SO2 sym st), 979. ¹H-NMR (500 MHz, Methanol-d₄) δ 8.63 (s, 1H), 7.83 (dd, J=6.7, 2.7 Hz, 1H), 7.78 (s, 1H), 7.62-7.53 (m, 1H), 7.24 (t, J=8.9 Hz, 1H), 7.20-7.06 (m, 4H), 7.00 (d, J=23.9 Hz, 1H), 4.30 (dd, J=8.4, 5.2 Hz, 1H), 4.20-4.09 (m, 2H), 3.92 (s, 3H), 3.32 (t, J=6.4 Hz, 2H), 3.24 (p, J=1.7 Hz, 1H), 2.44-2.28 (m, 2H), 2.19-2.07 (m, 1H), 2.00 (q, J=6.3 Hz, 2H), 1.97-1.82 (m, 1H). ¹³C-NMR (126 MHz, Methanol-d₄) δ 176.4, 175.7, 159.9, 159.1, 159.0, 158.4, 157.5, 156.4, 152.0, 149.5, 140.6, 136.8, 135.0, 132.2, 129.6, 127.7, 125.7, 125.7, 121.8, 121.6, 119.7, 117.8, 117.6, 108.7, 104.5, 100.4, 68.9, 67.2, 57.4, 53.4, 49.5, 49.5, 49.3, 49.2, 49.1, 49.0, 48.8, 48.7, 48.5, 39.1, 31.1, 30.3, 28.7. HRMS (ESI) m z calcd for C₃₂H₃₃ClFN₆O₉ ([M+H]⁺) 699.1982; found (rel int) 699.2000 ([M+H]⁺, 100).

### Example 2. Exemplary Biological Activity of a Prodrug Compound of the Present Technology

Cloning and Generation of Retroviral Vectors and Cell Lines

All HEK293t cell lines were generated using retroviral transduction with the MMLV gamma retroviral vector pLGPW or pLHCX (gifts from the Tortorella lab at Icahn School of Medicine at Mount Sinai). All CAR-T and SEAKER vectors were generated by cloning into the SFG gammaretroviral vector encoding CD19-directed CAR with human 4-1bb costimulatory element and CD3 zeta chain (SFG-19BBz). Standard molecular biology techniques and Gibson assembly were used to generate all constructs. Retroviral producer lines were generated with CaCl₂) (Promega) to transiently transfect H29 cells with retroviral constructs encoding CARs or SEAKERs. Supernatant from the H29 cells was collected and used to transduce 293Glv9 or PG13 stable packaging cells. Individual producers were subcloned and expanded.

Cell Culture

All cells were maintained in RPMI supplemented with 10% FBS, 2 mM L-glutamine, 100 IU/mL penicillin, 100 μg/mL nonessential amino acids, sodium pyruvate, N-2-hydroxyethylpiperazine-N-2-ethane sulfonic acid (HEPES), and human T cell media was supplemented with 100 IU/mL IL-2.

T Cell Isolation and Modification

Peripheral blood mononuclear cells (PBMCs) were isolated from healthy donors. PBMCs were activated with 50 ng/mL OKT-3 antibody (MACS) and 100 IU/mL IL2 for two days prior to transduction and maintained in 100 IU/mL thereafter. Transduction was performed by centrifugation of activated T cells in media from retroviral producers at 2000×g at room temperature for 1 hr on RetroNectin-coated plates (Takeda) for two consecutive days. Experiments were performed in compliance with all relevant ethical regulations and in accordance with TRB 06-107 and 16-312.

Flow Cytometry

Transduction efficiency was determined by flow cytometry using an Alexa647-labeled anti-idiotype antibody directed to the CD19-targeted CAR (mAb clone #19E3-generated at Memorial Sloan Kettering Cancer Center Antibody and Bioresource Core Facility). The following antibodies were used in flow cytometry experiments: Alexa647-anti-HA (Thermo—clone 26187, cat #26183-A647), APC-CD19 (BD—cat #555415), PE-CD69 (BioLegend cat #310906), APC/Cy70-CD3 (BioLegend cat #UCHT1). All samples were washed and stained in FACS buffer (2% FBS in PBS) at 4° C. Data was collected using a Guava EasyCyte HT flow cytometer (Millipore) or an LSR Fortessa (BD). Flow Jo software was used for all data analyses.

Immunoprecipitation and Western Blot Analysis

Anti-HA agarose beads (Thermo cat #26181) were incubated with cell supernatant or mouse ascites 2 hr at 4° C. on a nutator. The beads were washed 2× with cold PBS, and Laemmli sample buffer (BioRad Cat #161-0747)+/−Beta-mercaptoethanol (BME) was added. Protein samples (Immunoprecipitate or Total Cell Lysates) were scratched and heated 3× for 3 min at 95° C. and resolved by SDS-PAGE. Gels were transferred to nitrocellulose membranes and blotted for respective antibodies in TBST (Thermo cat #28360). Detection of antibody was achieved with Pierce ECL femto western substrate (Thermo cat #34095). The following antibodies were used for immunoblot: anti-Ms HRP (R&D systems HAF007), anti-Rb (R&D systems HAF008), anti-HA (Invitrogen cat. 26183). Polyclonal anti-CPG2 antibodies were raised in rabbits by inoculation with whole recombinant protein produced in E. coli and purified by nickel bead affinity chromatography (service performed by GenScript).

ELISA Analysis

Sandwich ELISAS were performed on 96-well Immulon HBX plates (Thermo). A mouse anti-HA antibody was used to capture protein (Invitrogen cat. 26183) and a polyclonal mouse anti-rabbit HRP antibody was used as detection antibody (R&D systems HAF008). Protein was detected using TMB substrate (Thermo cat #34028) and H₂SO₄ acid quench and read on a SpectraMax M2 plate reader (Molecular Devices). Data were analyzed with SoftMax Pro software.

Nitrocefin Cleavage Analysis

Cell supernatant, mouse ascites or blood was serially diluted (2-fold) and mixed 1:1 with 0.2 mM Nitrocefin (abcam cat # ab145625). Samples were incubated 1-16 hr at room temperature and absorbance at 490 nm was read on a SpectraMax M2 plate reader (Molecular Devices). Data were analyzed with SoftMax Pro software.

Glutamate Release Assays

Recombinant CPG2 enzyme was incubated with prodrugs in CPG2 reaction buffer (1M TrisHCL, 2 mM ZnCl₂) for 2 hr at 37° C. and the enzyme/prodrug mixture was combined 1:1 with Amplex Red™ Glutamate Oxidase Assay mixture (Thermo cat # A12221). Following 30 min at 37° C. fluorescent emission at 590 nm was measured on a SpectraMax M2 plate reader (Molecular Devices). Data were analyzed with SoftMax Pro software.

Cytotoxicity Assays

Prodrug/Drug IC50 and trans and cis-toxicity assays with secreted enzyme were performed using Cell-Titer Glo (Promega). Cells were analyzed in triplicate wells of a 96-well dish and equivalent volume of Cell-Titer Glo reagent was added to each well. Following a 10-minute incubation at room temperature samples were transferred to White 96-well Optiplate (Perkin Elmer) and luminescence was measured on a SpectraMax M2 plate reader (Molecular Devices). Data were analyzed with SoftMax Pro software.

The cytotoxicity of CAR-T cells and SEAKER cells was determined by luciferase-based assays. Target cells (Raji and SKOV-3 cells expressing Firefly luciferase and GFP (FFL-GFP) were used as target cells. Effector and tumor target cells were cocultured in triplicate at the indicated E:T ratio using clear bottom, white 96-well assay plates (Corning cat #3903) with 5E4 target cells in a total volume of 200 μL. Target cells alone were plated at the same cell density to determine maximum luciferase activity. Cells were cocultured 4-18 hr, at which time d-Luciferin substrate (Gold Biotech cat #LUCK) was added at a final concentration of 0.5 μg/pL to each well. Emitted light was detected in a Wallac EnVision Multilabel reader (Perkin Elmer). Target lysis was determined as (1−(RLUsample)/(RLUmax))×100.

Mixed cell bystander toxicity assays were performed by incubating untransduced T cells or SEAKER cells with Raji and SET2 cells at 4:1:1 (CAR-T cells:Raji:SET2) ratio. Following 24 hrs of coculture prodrug was added and cells were cultured an additional 48 hrs. prior to analysis by flow cytometry. Detection of GFP and anti-CD19 staining (APC-CD19-(BD cat #555415)) delineated Raji versus SET2 versus CAR-T cells. Cell count was measured by acquiring cells for 30 seconds/well on a Guava EasyCyte flow cytometer and multiplying percentage of respective gates by total cells acquired.

gLuc-19BBz Reporter CAR-T Cell In-Vitro Analysis

Assays measuring proliferation of gLuc-19BBz CAR-T cells were performed by measuring emitted light following cleavage of Coelenterazine substrate (Prolume cat #3032). Cells were cocultured 4-18 hr, at which time Coelenterazine was added at a final concentration of 2.5 μM to each well. Emitted light was detected in a Wallac EnVision Multilabel reader (Perkin Elmer).

Methotrexate Cleavage Assays

Methotrexate (Accord healthcare) was incubated at a final concentration of 450 uM with recombinant CPG2 enzyme, CAR-T cells, or cell supernatant and incubated at 37° C. for 16 hr. Absorbance at 390 nm was recorded on a NanoDrop spectrophotometer (Thermo Scientific).

Recombinant Proteins

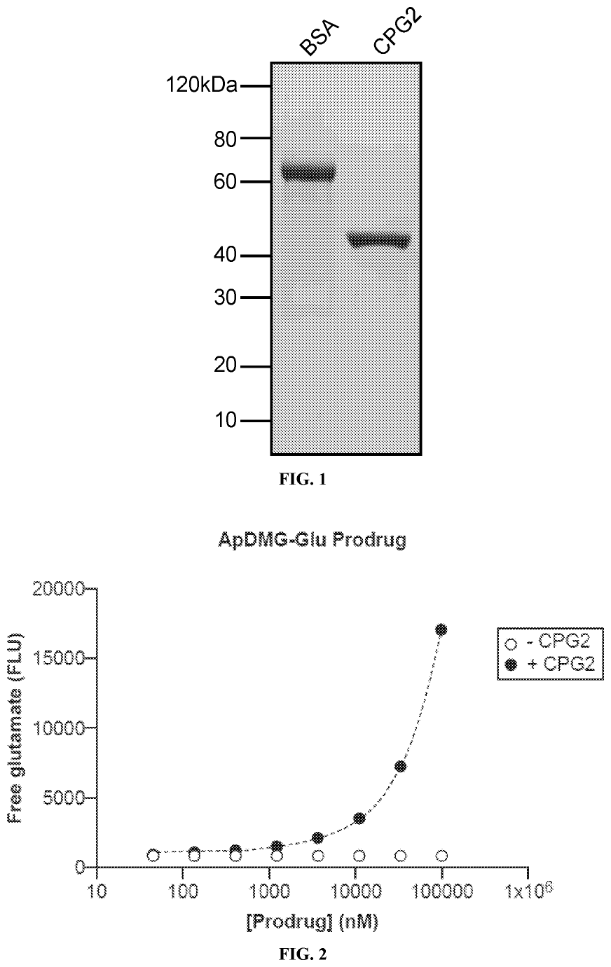

CPG2 proteins were produced and purified by GenScript. Constructs contain c-terminal HA and His epitope tags and were purified by nickel columns. FIG. 1 illustrates that recombinant Pseudomonas sp. CPG2 produced and purified from E. coli cell lysates migrates as a single band at ˜42 kDa by SDS-PAGE/coomassie.

Experiments in Mice

All experiments were performed in compliance with all relevant ethical regulations and in accordance with an Institutional Animal Care and Use Committee-approved protocol (protocol 96-11-044).

NSG mice (NOD.Cg-Prkdcscid Il2rgtm1Wjl/SzJ) were obtained from Jackson Laboratory. 7-13 week old mice were engrafted with 0.5E6 RajiGFP/Luc tumor cells (intraperitoneal) and treated 2 days later with 5E5 CAR-T cells (intraperitoneal). Prodrug was administered beginning at day 2 post CAR-T engraftment. For SKOV-3 experiments, 3E6 SKOV-3-CD19+ cells were engrafted IP and 3 days later mice were treated with 7.5E5 CAR-T cells (IP).

Bioluminescence Imaging

Bioluminescent tumor imaging was performed using a Xenogen IVIS imaging system with Living Image software (Xenogen Biosciences). Image acquisition was done on a 25-cm field of view at medium binning level at various exposure times. 100 μg Coelenterazine was administered IP for gLuc-CAR-T studies. 3 μg D-Luciferin was administered IP for Firefly-Luc tumor imaging. Images in a single data set were normalized together according to color intensity as indicated by scale bar.

Statistical Analysis

Log-rank and student's t-tests were performed using GraphPad Prism where appropriate. Statistical significance was indicated accordingly: *=p<0.05, **=p<0.01, ***=p<0.001.

Results and Comparative Compounds

Recombinant CPG2 protein was expressed and purified (see, then used to confirm cleavage of the glutamate moiety from the APdMG-Glu of Example 1 in an Amplex Red glutamate release assay (FIG. 2). Enzyme kinetic parameters were determined using a RapidFire-TOF-MS (time-of-flight mass spectrometry) assay (Table 1) and are presented alongside AMS-Glu of the SEAKER disclosures and methotrexate.

TABLE 1^{a}

| | | Vₘₐₓ | k_{cat} | Kₘ | k_{cat}/Kₘ |
|---|---|---|---|---|---|
| Enzyme | Analyte | (μM/s) | (s⁻¹) | (μM) | (μM⁻¹s⁻¹) |
| CPG2 | AMS-Glu | 0.21 ± | 0.27 ± | 409.90 ± | 0.00065 ± |
| | | 0.064 | 0.08 | 121.90 | 0.00003 |
| | ApDMG- | 0.10 ± | 0.137 ± | 2.25 ± | 0.066 ± |
| | Glu (S4) | 0.04 | 0.06 | 1.13 | 0.03 |
| | methotrexate | 0.39 ± | 10.36 ± | 9.45 ± | 1.30 ± |
| | | 0.05 | 1.47 | 4.72 | 0.62 |

^{a}Enzyme kinetics were assessed using a RapidFire-MS (mass spectrometry) assay for disappearance of substrate and/or appearance of product.

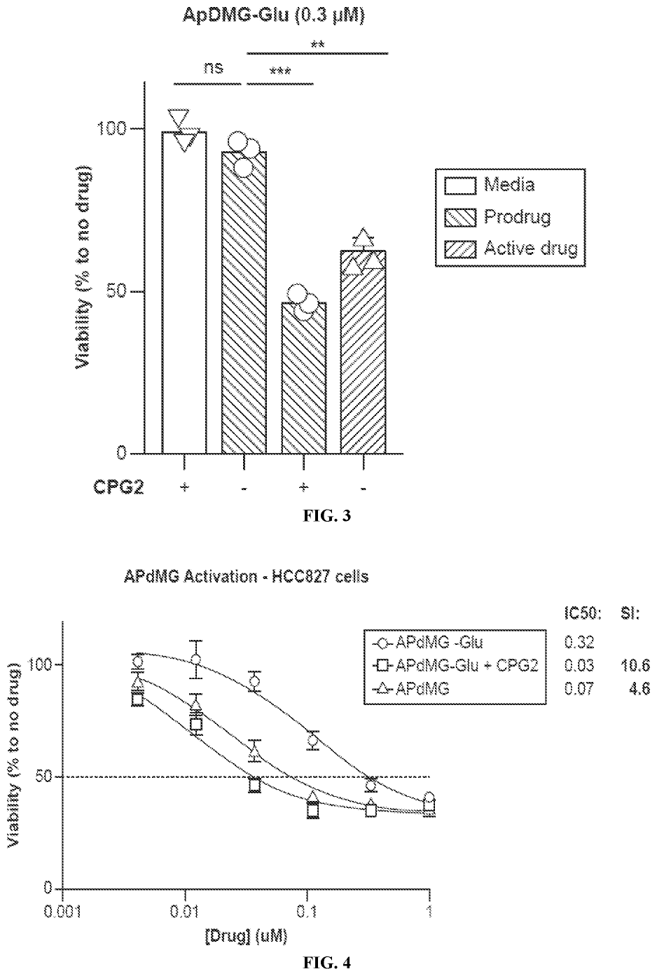

Treatment of HCC827 cells with APdMG-Glu plus recombinant CPG2, but not APdMG-Glu alone, resulted in cytotoxicity comparable to that of the parent drug gefitinib, consistent with effective activation of the prodrug by the CPG2 enzyme (FIG. 3). As illustrated in Table 2, APdMG-Glu exhibited selectivity for the exemplary cell types tested.

TABLE 2^{b}

| | Active | IC50 | | IC50 | Selective |
|---|---|---|---|---|---|
| Cell type | drug | (μM) | Prodrug | (μM) | index |
| HCC827 | APdMG | 0.02 | APdMG- | 0.11 | 5.5 |
| (adenocarcinoma) | | | Glu | | |
| PC9 | APdMG | 0.21 | APdMG- | 0.24 | 6 |
| (adenocarcinoma) | | | Glu | | |

^{b}IC50 values were assessed by Cell-Titer Glo analysis following 48 hours of drug treatment at ≥ 6 drug dilutions (3-fold).

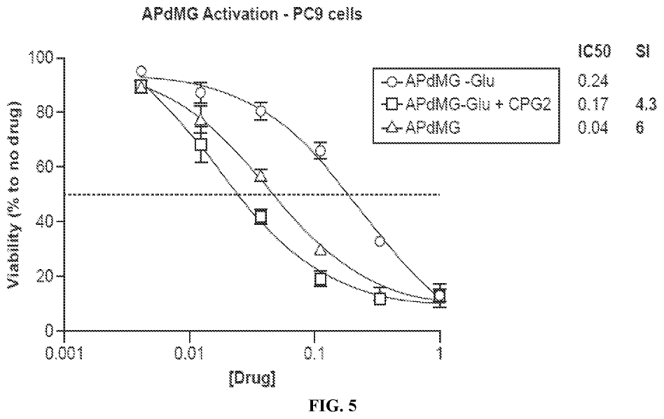

Additional IC₅₀ and selectivity data for APdMG-Glu with HCC827 cells (FIG. 4) and with PC9 cells (FIG. 5) further confirmed the viability of the present technology in the treatment methods.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A prodrug compound of Formula I

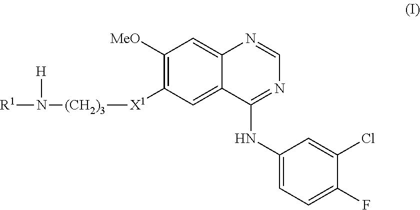

or a pharmaceutically acceptable salt and/or solvate thereof, wherein
X¹ is O or NH;
R¹ is

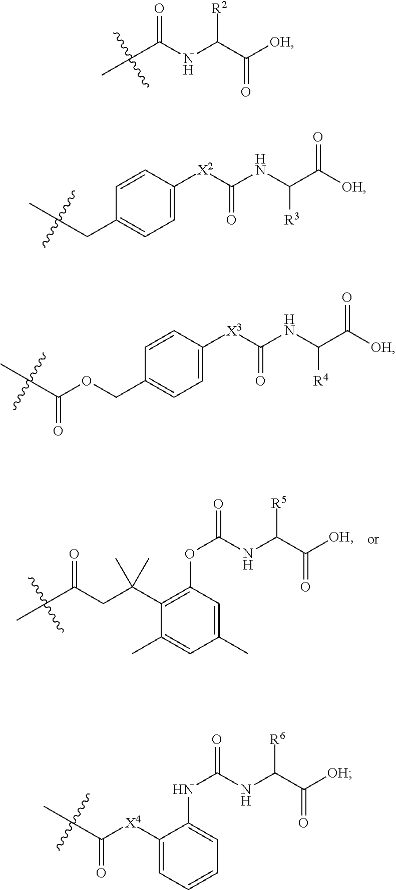

X² and X³ are each independently O or NH;
X⁴ is O, NH, CH₂, or C(Me)₂; and
R², R³, R⁴, R⁵, and R⁶ are each independently H, —(CH₂)₃—NH(NH)—NH₂, —(CH₂)₃—NH₂, —(CH₂)₄—NH₂, —CH₂—C(O)OH, —(CH₂)₂—C(O)OH,

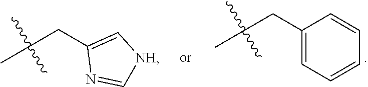 [SMILES: *Cc1c[nH]cn1 ; *Cc1ccccc1]

B. The prodrug compound of Paragraph A, wherein R is

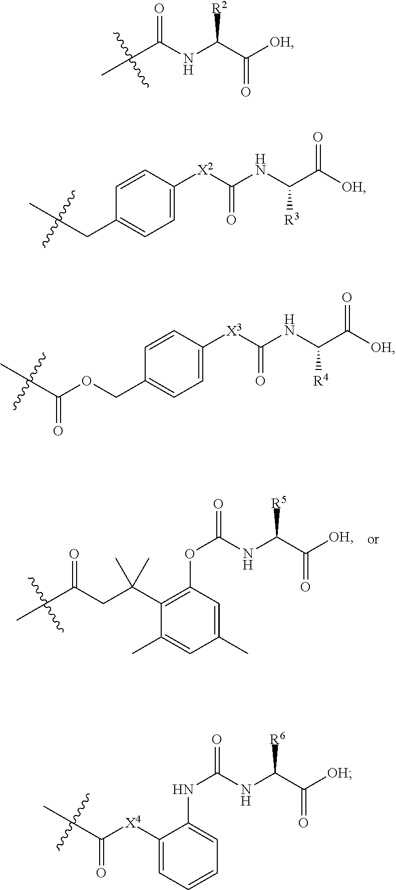

X² and X³ are each independently O or NH;
X⁴ is O, NH, CH₂, or C(Me)₂; and
R², R³, R⁴, R⁵, and R⁶ are each independently —(CH₂)₃—NH(NH)—NH₂, —(CH₂)₃—NH₂, —(CH₂)₄—NH₂, —CH₂—C(O)OH, —(CH₂)₂—C(O)OH,

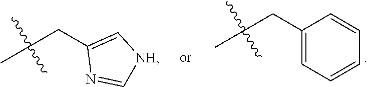 [SMILES: *Cc1c[nH]cn1 ; *Cc1ccccc1]

C. The prodrug compound of Paragraph A or Paragraph B, where R¹ is

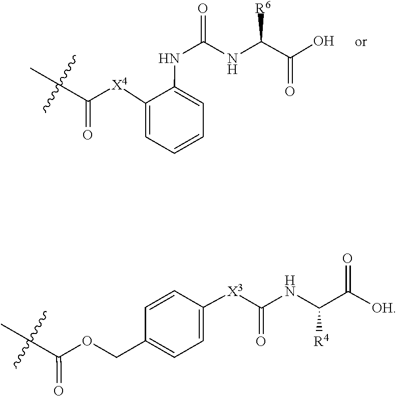

D. The prodrug compound of any one of Paragraphs A-C, wherein R¹ is

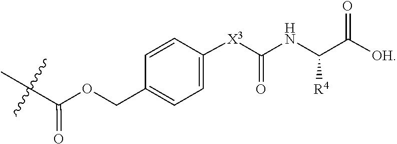

E. The prodrug compound of any one of Paragraphs A-D, wherein X¹ is O.
F. The prodrug compound of any one of Paragraphs A-E, wherein W¹ is NH.
G. The prodrug compound of any one of Paragraphs A-F, wherein the compound is

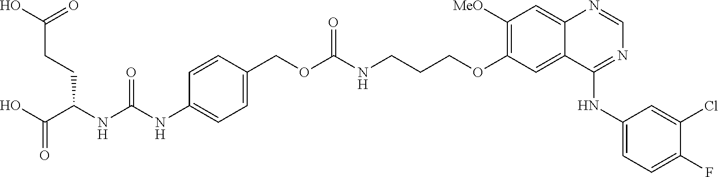 [SMILES: COc1cc2ncnc(Nc3ccc(F)c(Cl)c3)c2cc1OCCCNC(=O)OCc1ccc(NC(=O)N[C@@H](CCC(=O)O)C(=O)O)cc1]

or a pharmaceutically acceptable salt and/or solvate thereof.
H. A composition comprising a prodrug compound of any one of Paragraphs A-G and a pharmaceutically acceptable carrier.
I. A pharmaceutical composition comprising an effective amount of a prodrug compound of any one of Paragraphs A-G for treating cancer in a subject, and a pharmaceutically acceptable carrier.
J. The pharmaceutical composition of Paragraph I, wherein the pharmaceutical composition is formulated for use with an effective amount of an engineered immune cell; wherein the engineered immune cell comprises a prodrug converting enzyme and/or a nucleic acid encoding the prodrug converting enzyme and a receptor that binds to a target antigen and/or nucleic acid encoding the receptor.
K. The pharmaceutical composition of Paragraph J, wherein the pharmaceutical composition is formulated such that the prodrug compound is converted to an active drug by the prodrug converting enzyme.
L. The pharmaceutical composition of Paragraph J or Paragraph K, wherein the prodrug converting enzyme is a carboxypeptidase.
M. The pharmaceutical composition of any one of Paragraphs J-L, wherein the prodrug converting enzyme is CPG2.
N. A method for treating cancer in a subject in need thereof, the method comprising administering an effective amount of an engineered immune cell and administering an effective amount of a prodrug compound of any one of Paragraphs A-G, wherein the engineered immune cell comprises a prodrug converting enzyme and/or a nucleic acid encoding the prodrug converting enzyme and a receptor that binds to a target antigen and/or nucleic acid encoding the receptor.
O. The method of Paragraph N, wherein the prodrug compound is converted to an active drug by the prodrug converting enzyme.
P. The method of Paragraph N or Paragraph O, wherein the prodrug is administered subsequent to administration of the engineered immune cells.
Q. The method of any one of Paragraphs N-P, wherein the engineered immune cells are administered intravenously, intraperitoneally, subcutaneously, intramuscularly, or intratumorally.
R. The method of any one of Paragraphs N-Q, wherein the cancer or tumor is a carcinoma, sarcoma, a melanoma, or a hematopoietic cancer.
S. The method of any one of Paragraphs N-R, wherein the cancer or tumor is selected from among adrenal cancers, bladder cancers, blood cancers, bone cancers, brain cancers, breast cancers, epithelial carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, non small cell lung cancer, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, cancers with activated epidermal growth factor receptor's (EGFR) tyrosine kinase domain receptors, cancers with other activated receptor tyrosine kinases, and metastases of any one or more thereof.
T. The method of any one of Paragraphs N-S, wherein the method further comprises administering an additional cancer therapy.
U. The method of Paragraph T, wherein the additional cancer therapy is selected from among chemotherapy, radiation therapy, immunotherapy, monoclonal antibodies, anti-cancer nucleic acids or proteins, anti-cancer viruses or microorganisms, and any combinations thereof.
V. The method of any one of Paragraphs N-U, wherein the method further comprises administering a cytokine to the subject.
W. The method of Paragraph V, wherein the cytokine is administered prior to, during, or subsequent to administration of the one or more engineered immune cells.
X. The method of Paragraph V or Paragraph W, wherein the cytokine is selected from a group consisting of interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.
Y. The method of any one of Paragraphs N-X, wherein the prodrug converting enzyme is a carboxypeptidase.
Z. The method of any one of Paragraphs N-Y, wherein the prodrug converting enzyme is CPG2.
AA. The method of any one of Paragraphs N-Z, wherein the method does not comprise use of recombinant enzymes.
AB. The method of any one of Paragraphs N-AA, wherein the method comprises the use of recombinant enzymes.

Other embodiments are set forth in the following claims, along with the full scope of equivalents to which such claims are entitled.

1. A prodrug compound of Formula I 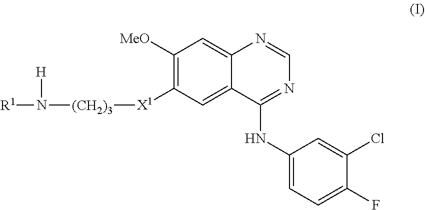
or a pharmaceutically acceptable salt and/or solvate thereof, wherein
X¹ is O or NH;
R¹ is 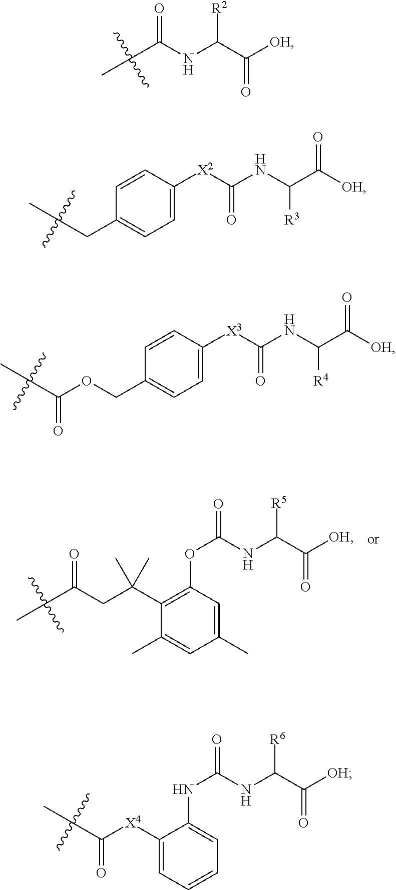
X² and X³ are each independently O or NH;
X⁴ is O, NH, CH₂, or C(Me)₂; and
R², R³, R⁴, R⁵, and R⁶ are each independently H, —(CH₂)₃—NH(NH)—NH₂, —(CH₂)₃—NH₂, —(CH₂)₄—NH₂, —CH₂—C(O)OH, —(CH₂)₂—C(O)OH,

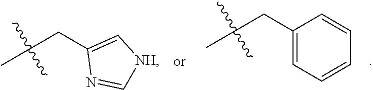 [SMILES: *Cc1c[nH]cn1 ; *Cc1ccccc1]

2. The prodrug compound of claim 1, wherein R¹ is 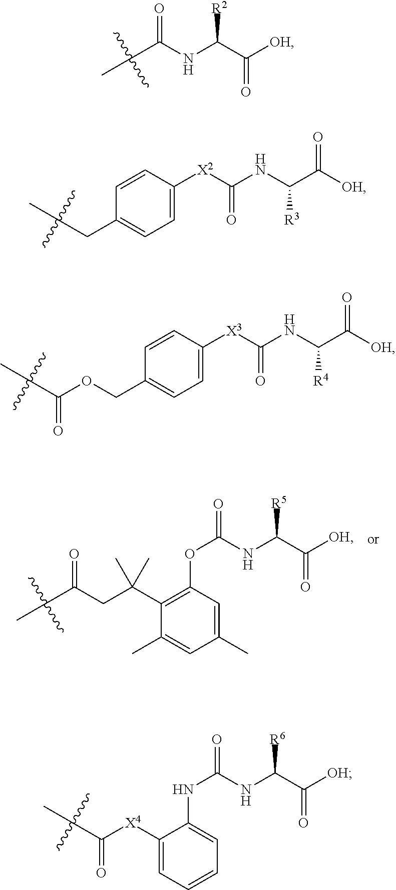
X² and X³ are each independently O or NH;
X⁴ is O, NH, CH₂, or C(Me)₂; and
R², R³, R⁴, R⁵, and R⁶ are each independently —(CH₂)₃—NH(NH)—NH₂, —(CH₂)₃—NH₂, —(CH₂)₄—NH₂, —CH₂—C(O)OH, —(CH₂)₂—C(O)OH,

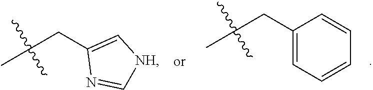 [SMILES: *Cc1c[nH]cn1 ; *Cc1ccccc1]

3. The prodrug compound of claim 1, where R¹ is

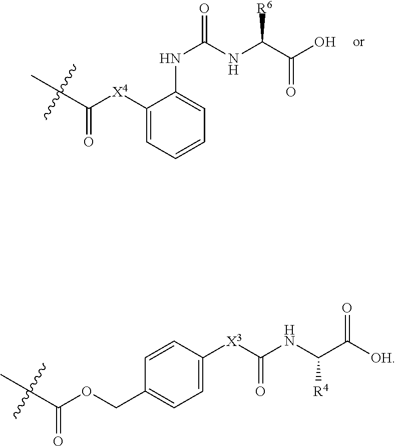

4. The prodrug compound of claim 1, wherein R¹ is

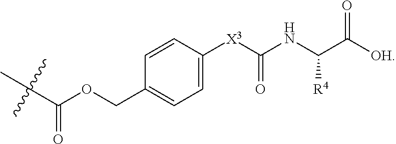

5. The prodrug compound of claim 1, wherein X¹ is O.

6. (canceled)

7. The prodrug compound of claim 1, wherein the compound is 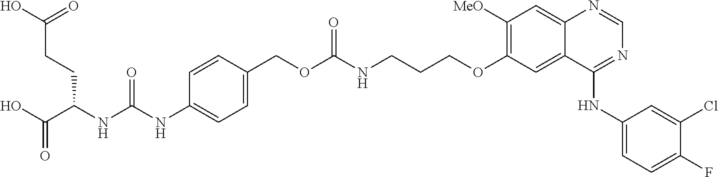 [SMILES: COc1cc2ncnc(Nc3ccc(F)c(Cl)c3)c2cc1OCCCNC(=O)OCc1ccc(NC(=O)N[C@@H](CCC(=O)O)C(=O)O)cc1]
or a pharmaceutically acceptable salt and/or solvate thereof.

8. A composition comprising a prodrug compound of claim 1 and a pharmaceutically acceptable carrier.

9. A pharmaceutical composition comprising an effective amount of a prodrug compound of claim 1 for treating cancer in a subject, and a pharmaceutically acceptable carrier.

10. The pharmaceutical composition of claim 9, wherein the pharmaceutical composition is formulated for use with an effective amount of an engineered immune cell; wherein the engineered immune cell comprises a prodrug converting enzyme and/or a nucleic acid encoding the prodrug converting enzyme, and a receptor that binds to a target antigen and/or nucleic acid encoding the receptor.

11. The pharmaceutical composition of claim 10, wherein the pharmaceutical composition is formulated such that the prodrug compound is converted to an active drug by the prodrug converting enzyme.

12.-17. (canceled)

18. A method for treating cancer in a subject in need thereof, the method comprising administering an effective amount of an engineered immune cell and administering an effective amount of a prodrug compound of claim 1, wherein the engineered immune cell comprises a prodrug converting enzyme and/or a nucleic acid encoding the prodrug converting enzyme, and a receptor that binds to a target antigen and/or nucleic acid encoding the receptor.

19. The method of claim 19, wherein the prodrug compound is converted to an active drug by the prodrug converting enzyme.

20. The method of claim 19, wherein the prodrug is administered subsequent to administration of the engineered immune cells.

21. (canceled)

22. The method of claim 19, wherein the cancer or tumor is a carcinoma, sarcoma, a melanoma, or a hematopoietic cancer.

23. The method of claim 19, wherein the cancer or tumor is selected from among adrenal cancers, bladder cancers, blood cancers, bone cancers, brain cancers, breast cancers, epithelial carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, non small cell lung cancer, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, cancers with activated epidermal growth factor receptor's (EGFR) tyrosine kinase domain receptors, cancers with other activated receptor tyrosine kinases, and metastases of any one or more thereof.

24.-25. (canceled)

26. The method of claim 19, wherein the method further comprises administering a cytokine to the subject.

27. The method of claim 26, wherein the cytokine is administered prior to, during, or subsequent to administration of the one or more engineered immune cells.

28. The method of claim 26, wherein the cytokine is selected from a group consisting of interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

29.-30. (canceled)

31. The method of claim 19, wherein the method does not comprise use of recombinant enzymes.

32. The method of claim 19, wherein the method comprises use of recombinant enzymes.
